(19) United States
(12) Patent Application Publication
(10) Pub. No.: US 2009/0253719 A1
(43) Pub. Date: Oct. 8, 2009

(54) CRYSTALLINE SALT FORMS OF ANTIFOLATE COMPOUNDS AND METHODS OF MANUFACTURING THEREOF

(75) Inventors: Harish K. Pimplaskar, Charlotte, NC (US); Mikhail Lebedev, Sodertalje (SE); Karol Horvath, Sodertalje (SE)

Correspondence Address: ALSTON & BIRD LLP, BANK OF AMERICA PLAZA, 101 SOUTH TRYON STREET, SUITE 4000, CHARLOTTE, NC, 28280-4000, US

(73) Assignee: Chelsea Therapeutics, Inc.

(21) Appl. No.: 12/419,732

(22) Filed: Apr. 7, 2009

Related U.S. Application Data
(60) Provisional application No. 61/042,998, filed on Apr. 7, 2008.
(60) Provisional application No. 61/042,994, filed on Apr. 7, 2008.

(51) Int. Cl. A61K 31/517; C07D 239/72; A61P 35/00

(52) U.S. Cl. 514/266.4; 544/291

(57) ABSTRACT

The present invention provides methods of preparing antifolate compounds. The inventive methods can particularly be use for preparing compounds exhibiting improved bioavailability, making the compound particularly useful in pharmaceutical compositions. The compounds prepared according to the inventive methods are useful in the treatment of multiple conditions, including abnormal cell proliferation, inflammatory diseases, asthma, and arthritis.

### CROSS-REFERENCE TO RELATED APPLICATIONS

The present application claims priority to U.S. Provisional Patent Application No. 61/042,998, filed Apr. 7, 2008, and U.S. Provisional Patent Application No. 61/042,994, filed Apr. 7, 2008, both of which are incorporated herein by reference in their entirety.

### FIELD OF THE INVENTION

The present application is directed to crystalline salt forms of pharmaceutically active compounds and methods of manufacturing such compounds. More specifically, the methods are useful for manufacturing antifolate compounds, particularly enantiomerically pure compounds.

### BACKGROUND

Folic acid is a water-soluble B vitamin known by the systematic name N-[4(2-amino-4-hydroxy-pteridin-6-ylmethylamino)-benzoyl]-L(+)-glutamic acid and having the structure provided below in Formula (1).

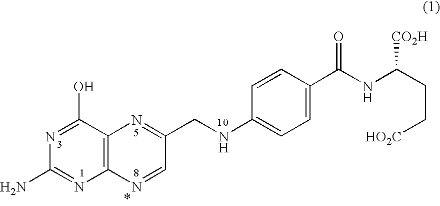

As seen in Formula (1), the folic acid structure can generally be described as being formed of a pteridine ring, a para-aminobenzoic acid moiety, and a glutamate moiety. Folic acid and its derivatives are necessary for metabolism and growth, particularly participating in the body's synthesis of thymidylate, amino acids, and purines. Derivatives of folic acid, such as naturally occurring folates, are known to have biochemical effects comparable to folic acid. Folic acid is known to be derivatized via hydrogenation, such as at the 1,4-diazine ring, or being methylated, formaldehydylated, or bridged, wherein substitution is generally at the N⁵ or N¹⁰ positions. Folates have been studied for efficacy in various uses including reduction in severity or incidence of birth defects, heart disease, stroke, memory loss, and age-related dementia.

Antifolate compounds, like folates, are structurally similar to folic acid; however, antifolate compounds function to disrupt folic acid metabolism. A review of antifolates is provided by Takamoto (1996) The Oncologist, 1:68-81, which is incorporated herein by reference. One specific group of antifolates, the so-called “classical antifolates,” is characterized by the presence of a folic acid p-aminobenzoylglutamic acid side chain, or a derivative of that side chain. Another group of antifolates, the so-called “nonclassical antifolates,” are characterized by the specific absence of the p-aminobenzoylglutamic group. Because antifolates have a physiological effect that is opposite the effect of folic acid, antifolates have been shown to exhibit useful physiological functions, such as the ability to destroy cancer cells by causing apoptosis.

Folate monoglutamylates and antifolate monoglutamylates are transported through cell membranes either in reduced form or unreduced form by carriers specific to those respective forms. Expression of these transport systems varies with cell type and cell growth conditions. After entering cells most folates, and many antifolates, are modified by polyglutamylation, wherein one glutamate residue is linked to a second glutamate residue at the a carboxy group via a peptide bond. This leads to formation of poly-L-γ-glutamylates, usually by addition of three to six glutamate residues. Enzymes that act on folates have a higher affinity for the polyglutamylated forms. Therefore, polyglutamylated folates generally exhibit a longer retention time within the cell.

An intact folate enzyme pathway is important to maintain de novo synthesis of the building blocks of DNA, as well as many important amino acids. Antifolate targets include the various enzymes involved in folate metabolism, including (i) dihydrofolate reductase (DHFR); (ii) thymidylate synthase (TS); (iii) folylpolyglutamyl synthase; and (iv) glycinamide ribonucleotide transformylase (GARFT) and aminoimidazole carboxamide ribonucleotide transformylase (AICART).

The reduced folate carrier (RFC), which is a transmembrane glycoprotein, plays an active role in the folate pathway transporting reduced folate into mammalian cells via the carrier mediated mechanism (as opposed to the receptor mediated mechanism). The RFC also transports antifolates, such as methotrexate. Thus, mediating the ability of RFC to function can affect the ability of cells to uptake reduced folates.

Polyglutamylated folates can function as enzyme cofactors, whereas polyglutamylated antifolates generally function as enzyme inhibitors. Moreover, interference with folate metabolism prevents de novo synthesis of DNA and some amino acids, thereby enabling antifolate selective cytotoxicity. Methotrexate, the structure of which is provided in Formula (2), is one antifolate that has shown use in cancer treatment, particularly treatment of acute leukemia, non-Hodgkin's lymphoma, breast cancer, head and neck cancer, choriocarcinoma, osteogenic sarcoma, and bladder cancer.

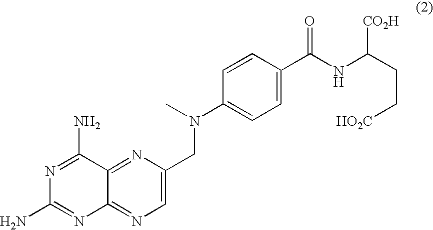

Nair et al. (J. Med. Chem. (1991) 34:222-227), incorporated herein by reference, demonstrated that polyglutamylation of classical antifolates was not essential for anti-tumor activity and may even be undesirable in that polyglutamylation can lead to a loss of drug pharmacological activity and target specificity. This was followed by the discovery of numerous nonpolyglutamylatable classical antifolates. See Nair et al. (1998) Proc. Amer. Assoc. Cancer Research 39:431, which is incorporated herein by reference. One particular group of nonpolyglutamylatable antifolates are characterized by a methylidene group (i.e., a ═CH₂ substituent) at the 4-position of the glutamate moiety. The presence of this chemical group has been shown to affect biological activity of the antifolate compound. See Nair et al. (1996) Cellular Pharmacology 3:29, which is incorporated herein by reference.

Further folic acid derivatives have also been studied in the search for antifolates with increased metabolic stability allowing for smaller doses and less frequent patient administration. For example, a dideaza (i.e., quinazoline-based) analog has been shown to avoid physiological hydroxylation on the pteridine ring system. Furthermore, replacement of the secondary amine nitrogen atom with an optionally substituted carbon atom has been shown to protect neighboring bonds from physiological cleavage.

One example of an antifolate having carbon replacement of the secondary amine nitrogen is 4-amino-4-deoxy-10-deazapteroyl-γ-methyleneglutamic acid—more commonly referred to as MDAM—the structure of which is provided in Formula (3).

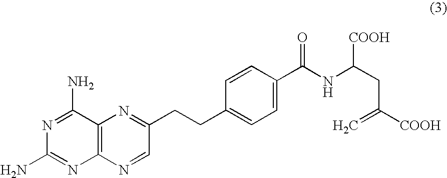

The L-enantiomer of MDAM has been shown to exhibit increased physiological activity. See U.S. Pat. No. 5,550,128, which is incorporated herein by reference. Another example of a classical antifolate designed for metabolic stability is ZD1694, which is shown in Formula (4).

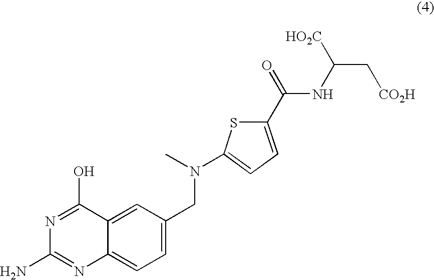

A group of antifolate compounds according to the structure shown in Formula (5) combines several of the molecular features described above, and this group of compounds is known by the names MobileTrexate, Mobile Trex, Mobiltrex, or M-Trex.

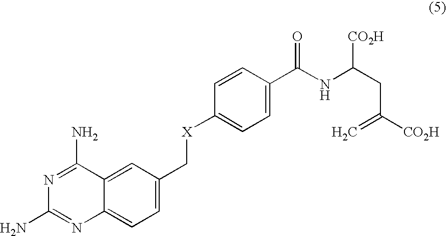

As shown in Formula (5), M-Trex encompasses a group of compounds wherein X can be CH₂, CHCH₃, CH(CH₂CH₃), NH, or NCH₃.

The effectiveness of antifolates as pharmaceutical compounds arises from other factors in addition to metabolic inertness, as described above. The multiple enzymes involved in folic acid metabolism within the body present a choice of inhibition targets for antifolates. In other words, it is possible for antifolates to vary as to which enzyme(s) they inhibit. For example, some antifolates inhibit primarily dihydrofolate reductase (DHFR), while other antifolates inhibit primarily thymidylate synthase (TS), glycinamide ribonucleotide formyltransferase (GARFT), or aminoimidazole carboxamide ribonucleotide transformylase, while still other antifolates inhibit combinations of these enzymes.

In light of the usefulness of antifolates in treating a variety of conditions, there remains a need in the art for methods of preparing antifolate compounds, particularly antifolate compounds that can be effectively incorporated into pharmaceutical compositions that can safely and effectively deliver the antifolates to a patient in need of treatment.

### SUMMARY OF THE INVENTION

The present invention provides antifolate compounds in forms exhibiting improved and/or otherwise desirable properties, as well as methods of preparing antifolate compounds. The antifolate compounds prepared according to the inventive methods are preferentially in a form exhibiting excellent bioavailability and are thus particularly useful in pharmaceutical compositions. In specific embodiments, the antifolate compounds prepared by the inventive methods are in the form of a particularly desired enantiomer, such as the (S) enantiomer. In further embodiments, the antifolate compounds prepared by the inventive methods are in the form of salts. Such salts provide for improved solubility, particularly in lower pH ranges. The salt forms of the antifolate compounds are also beneficial for increasing the amount of the antifolate compounds that is made available for biological activity when administered orally. The antifolate compounds prepared by the methods of the invention are useful in the treatment of a variety of conditions including, but not limited to, abnormal cellular proliferation, asthma and other inflammatory diseases, and rheumatoid arthritis and other autoimmune diseases.

In one aspect, the invention provides antifolate compounds having particularly desired form and properties. In certain embodiments, the invention provides compounds according to Formula (8)

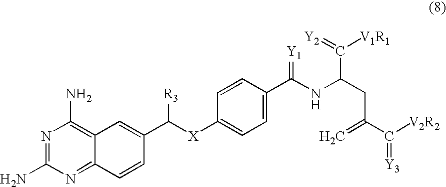

wherein:

X is CHR₈ or NR₈;

Y₁, Y₂, and Y₃ independently are O or S;

V₁ and V₂ independently are O, S, or NZ;

Z is H, optionally substituted alkyl, optionally substituted alkenyl, optionally substituted alkynyl, or alkaryl;

R₁ and R₂ independently are H, optionally substituted alkyl, optionally substituted alkenyl, optionally substituted alkynyl, or alkaryl;

R₃ is H, optionally substituted alkyl, optionally substituted alkenyl, optionally substituted alkynyl, optionally substituted alkoxy, hydroxyl, or halo; and

R₈ is H, optionally substituted alkyl, optionally substituted alkenyl, optionally substituted alkynyl, acyl, —C(O)-alkyl, —C(O)-alkenyl, or —C(O)-alkynyl as well as pharmaceutically acceptable esters, amides, salts, solvates, enantiomers, and prodrugs thereof.

In other embodiments, the invention is directed to antifolate compounds according to the following formula,

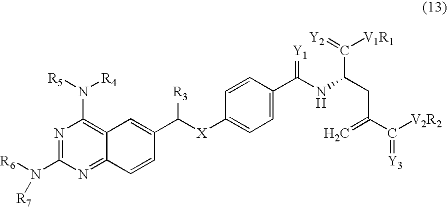

wherein:

X is CHR₈ or NR₈;

Y₁, Y₂, and Y₃ independently are O or S;

V₁ and V₂ independently are O, S, or NZ;

Z is H, optionally substituted alkyl, optionally substituted alkenyl, optionally substituted alkynyl, or alkaryl;

R₁ and R₂ independently are H, optionally substituted alkyl, optionally substituted alkenyl, optionally substituted alkynyl, or alkaryl;

R₃ is H, optionally substituted alkyl, optionally substituted alkenyl, optionally substituted alkynyl, optionally substituted alkoxy, hydroxyl, or halo; and

R₄, R₅, R₆, R₇, and R₈ independently are H, optionally substituted alkyl, optionally substituted alkenyl, optionally substituted alkynyl, acyl, —C(O)-alkyl, —C(O)-alkenyl, or —C(O)-alkynyl; or a pharmaceutically acceptable ester, amide, salt, solvate, enantiomer, or prodrug thereof;

wherein the compound is enantiomerically pure for the (S) enantiomer.

In further embodiments, the invention is directed to antifolate compounds according to the following formula

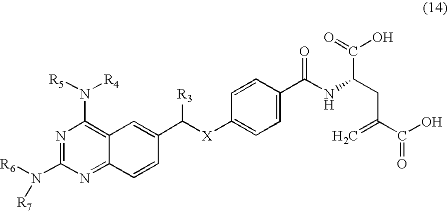

wherein:

X is CHR₈ or NR₈;

R₃ is H, optionally substituted alkyl, optionally substituted alkenyl, optionally substituted alkynyl, optionally substituted alkoxy, hydroxyl, or halo; and

R₄, R₅, R₆, R₇, and R₈ independently are H, optionally substituted alkyl, optionally substituted alkenyl, optionally substituted alkynyl, acyl, —C(O)-alkyl, —C(O)-alkenyl, or —C(O)-alkynyl; or a pharmaceutically acceptable ester, amide, salt, solvate, enantiomer, or prodrug thereof;

wherein the compound is enantiomerically pure for the (S) enantiomer.

In yet other embodiments, the invention is directed to antifolate compounds according to the following formula

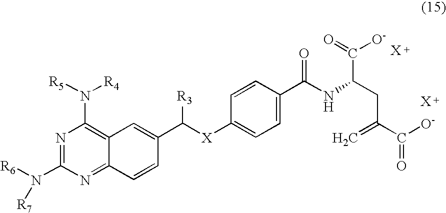

wherein:

X is CHR₈ or NR₈;

R₃ is H, optionally substituted alkyl, optionally substituted alkenyl, optionally substituted alkynyl, optionally substituted alkoxy, hydroxyl, or halo; and

R₄, R₅, R₆, R₇, and R₈ independently are H, optionally substituted alkyl, optionally substituted alkenyl, optionally substituted alkynyl, acyl, —C(O)-alkyl, —C(O)-alkenyl, or —C(O)-alkynyl; or a pharmaceutically acceptable ester, amide, solvate, enantiomer, or prodrug thereof;

wherein the compound is in the form of a crystalline salt that is enantiomerically pure for the (S) enantiomer, and wherein X⁺ is a counterion.

In specific embodiments, the invention is directed to antifolate compounds according to Formula (11).

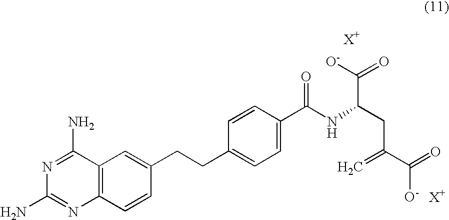

In preferred embodiments, the antifolate compound is in the form of a crystalline salt. For example, X⁺ can represent an alkali metal cation, particularly sodium or potassium. In other preferred embodiments, the antifolate compound is enantiomerically pure for the (S) enantiomer.

In specific embodiments, the invention provides an antifolate compound that is a crystalline, alkali metal salt of (S)-2-{4-[2-(2,4-diamino-quinazolin-6-yl)-ethyl]-benzoylamino}-4-methylene-pentanedioic acid, wherein the compound exhibits an enantiomeric purity for the (S) enantiomer of at least about 95%. The alkali metal particularly may be selected from the group consisting of sodium, potassium, and combinations thereof.

The invention also provides the compound (S)-2-{4-[2-(3,4-diamino-quinazolin-6-yl)-ethyl]benzolyamino}-4-methylene-pentanedioic acid disodium salt, wherein the compound is crystalline. Preferably, the compound exhibits an enantiomeric purity for the (S) enantiomer of at least about 95%.

Still further, the invention provides the compound (S)-2-{4-[2-(3,4-diamino-quinazolin-6-yl)-ethyl]benzolyamino}-4-methylene-pentanedioic acid dipotassium salt, wherein the compound is crystalline. Preferably, the compound exhibits an enantiomeric purity for the (S) enantiomer of at least about 95%.

In another aspect, the present invention provides methods for preparing antifolate compounds, such as those described above. In certain embodiments, the method generally comprises a method illustrated by Reaction Scheme I, which is provided herein. Particularly, the method can comprise the following steps:

1) Providing a starting reagent comprising a benzyl moiety with one or more substituents capable of being cyclized to a 2,4-disubstituted fused aromatic nitrogen-containing heterocycle and an addition site for a coupling reaction;

2) Reacting the starting reagent with a di-substituted benzyl moiety, wherein one substituent is capable of reacting to the free methyl group of the starting reagent and an optionally protected reacting group as a site for a later reaction;

3) Cyclizing the compound of Step 2) to form to a 2,4-disubstituted fused aromatic nitrogen-containing heterocycle coupled to a p-substituted benzyl moiety, the p-substituent being reactive with a group on an optionally substituted glutamate residue;

4) Reacting the compound of Step 3) with a methylene substituted glutamate residue that is further, optionally substituted; and

5) Optionally reacting the compound of Step 4) with reactants suitable to alter the state of the glutamate residue, such as to form an acid or a salt.

In certain embodiments, the method of the invention can comprise a step for forming an intermediate compound having a desired enantiomeric form. For example, in one embodiment, the method comprises the step of reacting 4-[2-(2,4-diamino-quinazolin-6-yl)-ethyl]-benzoic acid with (S)-2-amino-4-methylene-pentanedioic acid dimethyl ester hydrochloride to form an intermediate of (S)-2-{4-[2-(2,4-diamino-quinazolin-6-yl)-ethyl]-benzoylamino}-4-methylene-pentanedioic acid dimethyl ester.

In further embodiments, the method can comprise additional steps. For example, the method can further comprise reacting the intermediate (S)-2-{4-[2-(2,4-diamino-quinazolin-6-yl)-ethyl]-benzoylamino}-4-methylene-pentanedioic acid dimethyl ester with a base to form (S)-2-{4-[2-(2,4-diamino-quinazolin-6-yl)-ethyl]-benzoylamino}-4-methylene-pentanedioic acid. Still further, the method can comprise reacting the (S)-2-{4-[2-(2,4-diamino-quinazolin-6-yl)-ethyl]-benzoylamino}-4-methylene-pentanedioic acid with an alkali metal base to form (S)-2-{4-[2-(3,4-diamino-quinazolin-6-yl)-ethyl]benzolyamino}-4-methylene-pentanedioic acid, alkali metal salt. The antifolate compound is a crystalline salt that is enantiomerically pure for the (S) enantiomer.

In a specific embodiment, the invention provides yet another method for preparing an antifolate compound. Particularly, the compound is in the form of a crystalline salt that is enantiomerically pure for the (S) enantiomer. According to this embodiment, the method comprises: a) reacting 6-nitro-m toluic acid with triethylamine and iso-butyl chloroformate to form a reaction product; b) reacting the product from step a) with POCl₃ to form a second reaction product; c) reacting the product from step b) with 4-methoxycarbonylbenzaldehyde to form a third reaction product; d) reacting the product from step c) with hydrogen in the presence of a catalyst to form a fourth reaction product; e) reacting the product from step d) with chloroformamidine hydrochloride to form a fifth reaction product; f) reacting the product from step e) with hydrochloric acid to form a sixth reaction product; g) reacting the product from step f) with (S)-2-amino-4-methylene-pentanedioic acid dimethyl ester hydrochloride to form a seventh reaction product that is enantiomerically pure for the (S) enantiomer; h) reacting the product from step g) with sodium hydroxide to form an eighth reaction product; and i) reacting the product from step h) with an alkali metal base to form the antifolate compound according to the following formula

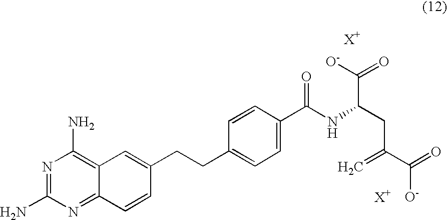

wherein X⁺ is an alkali metal cation, and the compound is in the form of a crystalline salt that is enantiomerically pure for the (S) enantiomer.

In other embodiments, the method of preparing antifolate compounds according to the invention includes the preparation of certain intermediate compounds. Preparation of such intermediates can provide multiple benefits. For example, formation of the intermediate can be useful as a purification step to isolate the reaction product and remove any impurities that are not isolated with the reaction product. This is particularly possible when the intermediate compound is a crystalline compound. For example, such a crystalline compound could be subjected to appropriate recrystallization methods to purify the product.

The formation of intermediate compounds is also useful for long term storage of reaction product and for maintaining the compound in a ready form for easy transformation into an antifolate compound. For example, in certain embodiments, the method of the invention provides for preparation of an intermediate compound in the form of a stable, crystalline compound. Such crystalline forms are particularly capable of exhibiting a long, stable shelf life.

In one embodiment, the method of the invention comprises the step of forming a stable, crystalline intermediate compound by reacting (S)-2-{4-[2-(2,4-diamino-quinazolin-6-yl)-ethyl]-benzoylamino}-4-methylene-pentanedioic acid with a suitable organic counterion, such as (R)-(+)-1-(2-naphthyl)ethylamine or a glutamic acid moiety.

In other embodiments, the present invention is directed to pharmaceutical compositions. In one embodiment, the invention provides a pharmaceutical composition comprising an antifolate compound according to the following formula

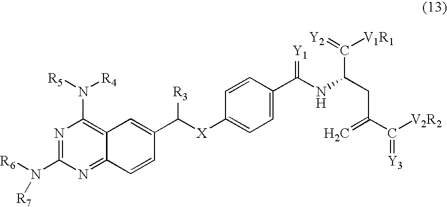

wherein:

X is CHR₈ or NR₈;

Y₁, Y₂, and Y₃ independently are O or S;

V₁ and V₂ independently are O, S, or NZ;

Z is H, optionally substituted alkyl, optionally substituted alkenyl, optionally substituted alkynyl, or alkaryl;

R₁ and R₂ independently are H, optionally substituted alkyl, optionally substituted alkenyl, optionally substituted alkynyl, or alkaryl;

R₃ is H, optionally substituted alkyl, optionally substituted alkenyl, optionally substituted alkynyl, optionally substituted alkoxy, hydroxyl, or halo; and

R₄, R₅, R₆, R₇, and R₈ independently are H, optionally substituted alkyl, optionally substituted alkenyl, optionally substituted alkynyl, acyl, —C(O)-alkyl, —C(O)-alkenyl, or —C(O)-alkynyl; or a pharmaceutically acceptable ester, amide, salt, solvate, enantiomer, or prodrug thereof. Preferably, the compound is enantiomerically pure for the (S) enantiomer. The compositions preferably further comprise a pharmaceutically acceptable carrier.

In still other embodiments, the invention provides methods of treatment. In particular, the invention may be directed to a method for treating a condition selected from the group consisting of abnormal cell proliferation, inflammation, asthma, and arthritis. The method can comprise administering to a subject in need of treatment a compound according to the following formula

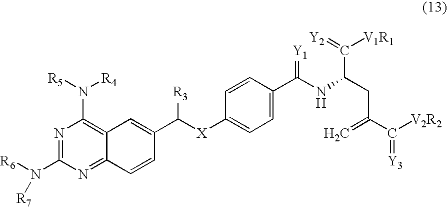

wherein:

X is CHR₈ or NR₈;

Y₁, Y₂, and Y₃ independently are O or S;

V₁ and V₂ independently are O, S, or NZ;

Z is H, optionally substituted alkyl, optionally substituted alkenyl, optionally substituted alkynyl, or alkaryl;

R₁ and R₂ independently are H, optionally substituted alkyl, optionally substituted alkenyl, optionally substituted alkynyl, or alkaryl;

R₃ is H, optionally substituted alkyl, optionally substituted alkenyl, optionally substituted alkynyl, optionally substituted alkoxy, hydroxyl, or halo; and

R₄, R₅, R₆, R₇, and R₈ independently are H, optionally substituted alkyl, optionally substituted alkenyl, optionally substituted alkynyl, acyl, —C(O)-alkyl, —C(O)-alkenyl, or —C(O)-alkynyl; or a pharmaceutically acceptable ester, amide, salt, solvate, enantiomer, or prodrug thereof. Preferably, the compound is enantiomerically pure for the (S) enantiomer. The methods may be carried with numerous compounds provided according to the present invention.

### BRIEF DESCRIPTION OF THE DRAWINGS

Having thus described the invention in general terms, reference will now be made to the accompanying drawing, which is not necessarily drawn to scale, and wherein:

FIG. 1 is an X-ray powder diffraction pattern graph of a salt compound according to one embodiment of the invention.

### DETAILED DESCRIPTION

The invention now will be described more fully hereinafter through reference to various embodiments. These embodiments are provided so that this disclosure will be thorough and complete, and will fully convey the scope of the invention to those skilled in the art. Indeed, the invention may be embodied in many different forms and should not be construed as limited to the embodiments set forth herein; rather, these embodiments are provided so that this disclosure will satisfy applicable legal requirements. As used in the specification, and in the appended claims, the singular forms “a”, “an”, “the”, include plural referents unless the context clearly dictates otherwise.

The present invention provides methods of preparing antifolate compounds. The antifolate compounds are particularly useful in pharmaceutical compositions. In preferred embodiments, the antifolate compounds are prepared in the form of salts, particularly alkali metal salts. The inventive methods provide antifolate compounds in forms that exhibit increased activity and bioavailability and that are useful in the treatment of a number of conditions and diseases, particularly for the treatment of abnormal cell proliferation, inflammation, arthritis, or asthma.

### I. DEFINITIONS

The term “metabolically inert antifolate” as used herein means compounds that are (i) folic acid analogs capable of disrupting folate metabolism and (ii) non-polyglutamylatable. In certain embodiments, the term can mean compounds that are also (iii) non-hydroxylatable.

The term “alkali metal” as used herein means Group IA elements and particularly includes sodium, lithium, and potassium; the term “alkali metal salt” as used herein means an ionic compound wherein the cation moiety of the compound comprises an alkali metal, particularly sodium, lithium, or potassium.

The term “alkyl” as used herein means saturated straight, branched, or cyclic hydrocarbon groups. In particular embodiments, alkyl refers to groups comprising 1 to 10 carbon atoms (“C₁₋₁₀ alkyl”). In further embodiments, alkyl refers to groups comprising 1 to 8 carbon atoms (“C₁₋₈ alkyl”), 1 to 6 carbon atoms (“C₁₋₆ alkyl”), or 1 to 4 carbon atoms (“C₁₋₄ alkyl”). In specific embodiments, alkyl refers to methyl, ethyl, propyl, isopropyl, cyclopropyl, butyl, isobutyl, t-butyl, pentyl, cyclopentyl, isopentyl, neopentyl, hexyl, isohexyl, cyclohexyl, cyclohexylmethyl, 3-methylpentyl, 2,2-dimethylbutyl, and 2,3-dimethylbutyl. Substituted alkyl refers to alkyl substituted with one or more moieties selected from the group consisting of halo (e.g., Cl, F, Br, and I); halogenated alkyl (e.g., CF₃, 2-Br-ethyl, CH₂F, CH₂Cl, CH₂CF₃, or CF₂CF₃; hydroxyl; amino; carboxylate; carboxamido; alkylamino; arylamino; alkoxy; aryloxy; nitro; azido; cyano; thio; sulfonic acid; sulfate; phosphonic acid; phosphate; and phosphonate.

The term “alkenyl” as used herein means alkyl moieties wherein at least one saturated C—C bond is replaced by a double bond. In particular embodiments, alkenyl refers to groups comprising 1 to 10 carbon atoms (“C₁₋₁₀ alkenyl”). In further embodiments, alkenyl refers to groups comprising 1 to 8 carbon atoms (“C₁₋₈ alkenyl”), 1 to 6 carbon atoms (“C₁₋₆ alkenyl”), or 1 to 4 carbon atoms (“C₁₋₄ alkenyl”). In specific embodiments, alkenyl can be vinyl, allyl, 1-propenyl, 2-propenyl, 1-butenyl, 2-butenyl, 3-butenyl, 1-pentenyl, 2-pentenyl, 3-pentenyl, 4-pentenyl, 1-hexenyl, 2-hexenyl, 3-hexenyl, 4-hexenyl, or 5-hexenyl. Substituted alkenyl refers to alkenyl substituted with one or more moieties selected from the group consisting of halo (e.g., Cl, F, Br, and I); halogenated alkyl (e.g., CF₃, 2-Br-ethyl, CH₂F, CH₂Cl, CH₂CF₃, or CF₂CF₃; hydroxyl; amino; carboxylate; carboxamido; alkylamino; arylamino; alkoxy; aryloxy; nitro; azido; cyano; thio; sulfonic acid; sulfate; phosphonic acid; phosphate; and phosphonate.

The term “alkynyl” as used herein means alkynyl moieties wherein at least one saturated C—C bond is replaced by a triple bond. In particular embodiments, alkynyl refers to groups comprising 1 to 10 carbon atoms (“C₁₋₁₀ alkynyl”). In further embodiments, alkynyl refers to groups comprising 1 to 8 carbon atoms (“C₁₋₈ alkynyl”), 1 to 6 carbon atoms (“C₁₋₆ alkynyl”), or 1 to 4 carbon atoms (“C₁₋₄ alkynyl”). In specific embodiments, alkynyl can be ethynyl, 1-propynyl, 2-propynyl, 1-butynyl, 2-butynyl, 3-butynyl, 1-pentynyl, 2-pentynyl, 3-pentynyl, 4-pentynyl, 1- hexynyl, 2-hexynyl, 3-hexynyl, 4-hexynyl, or 5-hexynyl. Substituted alkynyl refers to alkynyl substituted with one or more moieties selected from the group consisting of halo (e.g., Cl, F, Br, and I); halogenated alkyl (e.g., CF₃, 2-Br-ethyl, CH₂F, CH₂Cl, CH₂CF₃, or CF₂CF₃; hydroxyl; amino; carboxylate; carboxamido; alkylamino; arylamino; alkoxy; aryloxy; nitro; azido; cyano; thio; sulfonic acid; sulfate; phosphonic acid; phosphate; and phosphonate.

The term “alkoxy” as used herein means straight or branched chain alkyl groups linked by an oxygen atom (i.e., —O-alkyl), wherein alkyl is as described above. In particular embodiments, alkoxy refers to oxygen-linked groups comprising 1 to 10 carbon atoms (“C₁₋₁₀ alkoxy”). In further embodiments, alkoxy refers to oxygen-linked groups comprising 1 to 8 carbon atoms (“C₁₋₈ alkoxy”), 1 to 6 carbon atoms (“C₁₋₆ alkoxy”), or 1 to 4 carbon atoms (“C₁₋₄ alkoxy”). Substituted alkoxy refers to alkoxy substituted with one or more moieties selected from the group consisting of halo (e.g., Cl, F, Br, and I); halogenated alkyl (e.g., CF₃, 2-Br-ethyl, CH₂F, CH₂Cl, CH₂CF₃, or CF₂CF₃; hydroxyl; amino; carboxylate; carboxamido; alkylamino; arylamino; alkoxy; aryloxy; nitro; azido; cyano; thio; sulfonic acid; sulfate; phosphonic acid; phosphate; and phosphonate.

The term “halo” or “halogen” as used herein means fluorine, chlorine, bromine, or iodine.

The term “aryl” as used herein means a stable monocyclic, bicyclic, or tricyclic carbon ring of up to 8 members in each ring, wherein at least one ring is aromatic as defined by the Hückel 4n+2 rule. Exemplary aryl groups according to the invention include phenyl, naphthyl, tetrahydronaphthyl, and biphenyl. The aryl group can be substituted with one or more moieties selected from the group consisting of hydroxyl, amino, alkylamino, arylamino, alkoxy, aryloxy, nitro, cyano, sulfonic acid, sulfate, phosphonic acid, phosphate, or phosphonate.

The terms “aralkyl” and “arylalkyl” as used herein mean an aryl group as defined above linked to the molecule through an alkyl group as defined above.

The terms “alkaryl” and “alkylaryl” as used herein means an alkyl group as defined above linked to the molecule through an aryl group as defined above.

The term “acyl” as used herein means a carboxylic acid ester in which the non-carbonyl moiety of the ester group is selected from straight, branched, or cyclic alkyl or lower alkyl; alkoxyalkyl including methoxymethyl; aralkyl including benzyl; aryloxyalkyl such as phenoxymethyl; aryl including phenyl optionally substituted with halogen, C₁-C₆ alkyl or C₁-C₆ alkoxy; sulfonate esters such as alkyl or aralkyl sulphonyl including methanesulfonyl; mono-, di-, or triphosphate ester; trityl or monomethoxytrityl; substituted benzyl; trialkylsilyl such as dimethyl-t-butylsilyl or diphenylmethylsilyl. Aryl groups in the esters optimally comprise a phenyl group.

The term “amino” as used herein means a moiety represented by the structure NR₂, and includes primary amines, and secondary and tertiary amines substituted by alkyl (i.e., alkylamino). Thus, R₂ may represent two hydrogen atoms, two alkyl moieties, or one hydrogen atom and one alkyl moiety.

The terms “alkylamino” and “arylamino” as used herein mean an amino group that has one or two alkyl or aryl substituents, respectively.

The term “analogue” as used herein means a compound in which one or more individual atoms or functional groups have been replaced, either with a different atom or a different functional, generally giving rise to a compound with similar properties.

The term “derivative” as used herein means a compound that is formed from a similar, beginning compound by attaching another molecule or atom to the beginning compound. Further, derivatives, according to the invention, encompass one or more compounds formed from a precursor compound through addition of one or more atoms or molecules or through combining two or more precursor compounds.

The term “prodrug” as used herein means any compound which, when administered to a mammal, is converted in whole or in part to a compound of the invention.

The term “active metabolite” as used herein means a physiologically active compound which results from the metabolism of a compound of the invention, or a prodrug thereof, when such compound or prodrug is administered to a mammal.

The terms “therapeutically effective amount” or “therapeutically effective dose” as used herein are interchangeable and mean a concentration of a compound according to the invention, or a biologically active variant thereof, sufficient to elicit the desired therapeutic effect according to the methods of treatment described herein.

The term “pharmaceutically acceptable carrier” as used herein means a carrier that is conventionally used in the art to facilitate the storage, administration, and/or the healing effect of a biologically active agent.

The term “intermittent administration” as used herein means administration of a therapeutically effective dose of a composition according to the invention, followed by a time period of discontinuance, which is then followed by another administration of a therapeutically effective dose, and so forth.

The term “antiproliferative agent” as used herein means a compound that decreases the hyperproliferation of cells.

The term “abnormal cell proliferation” as used herein means a disease or condition characterized by the inappropriate growth or multiplication of one or more cell types relative to the growth of that cell type or types in an individual not suffering from that disease or condition.

The term “cancer” as used herein means a disease or condition characterized by uncontrolled, abnormal growth of cells, which can spread locally or through the bloodstream and lymphatic system to other parts of the body. The term includes tumor-forming or non-tumor forming cancers, and includes various types of cancers, such as primary tumors and tumor metastasis.

The term “tumor” as used herein means an abnormal mass of cells within a multicellular organism that results from excessive cell division that is uncontrolled and progressive, also called a neoplasm. A tumor may either be benign or malignant.

The term “fibrotic disorders” as used herein means fibrosis and other medical complications of fibrosis which result in whole or in part from the proliferation of fibroblasts.

Chemical nomenclature using the symbols “D” and “L” or “R” and “S” are understood to relate the absolute configuration, or three-dimensional arrangement, of atoms or groups around a chiral element, which may be a center, usually an atom, an axis, or a plane. As used herein, the “D/L” system and the “R/S” systems are meant to be used interchangeably such that “D” in the former system corresponds to “R” in the later system and “L” in the former system corresponds to “S” in the later system.

### II. Compounds

The methods of the invention are useful for the preparation of one or more antifolate compounds. In specific embodiments, the antifolate compounds prepared according to the invention are metabolically inert antifolates. As recognized in the art, antifolates are compounds that interfere with various stages of folate metabolism. Thus, the compounds prepared according to the methods of the invention can particularly be used in pharmaceutical compositions useful for the treatment of diseases and conditions related to or capable of being treated by disruption of folate metabolism, or other biological mechanisms related to folate metabolism. The compounds of the invention may be described according to a number of specific formulas, which are more particularly described below.

In one embodiment, the invention comprises antifolate compounds that may be prepared according to one or more of the synthesis methods described herein. In particular, the compounds can include those having the structure provided below in Formula (6),

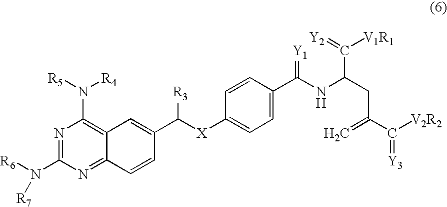

wherein:

X is CHR₈ or NR₈;

Y₁, Y₂, and Y₃ independently are O or S;

V₁ and V₂ independently are O, S, or NZ;

Z is H, optionally substituted alkyl, optionally substituted alkenyl, optionally substituted alkynyl, or alkaryl;

R₁ and R₂ independently are H, optionally substituted alkyl, optionally substituted alkenyl, optionally substituted alkynyl, or alkaryl;

R₃ is H, optionally substituted alkyl, optionally substituted alkenyl, optionally substituted alkynyl, optionally substituted alkoxy, hydroxyl, or halo; and

R₄, R₅, R₆, R₇, and R₈ independently are H, optionally substituted alkyl, optionally substituted alkenyl, optionally substituted alkynyl, acyl, —C(O)-alkyl, —C(O)-alkenyl, or —C(O)-alkynyl; as well as pharmaceutically acceptable esters, amides, salts, solvates, enantiomers, and prodrugs thereof.

In another embodiment, the invention comprises compounds that may be prepared according to the methods described herein and have the structure provided in Formula (7)

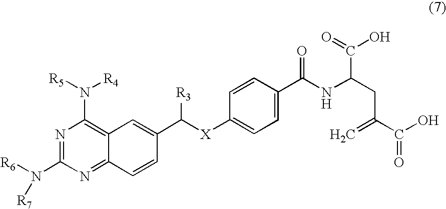

wherein:

X is CHR₈ or NR₈;

R₃ is H, optionally substituted alkyl, optionally substituted alkenyl, optionally substituted alkynyl, optionally substituted alkoxy, hydroxyl, or halo; and

R₄, R₅, R₆, R₇, and R₈ independently are H, optionally substituted alkyl, optionally substituted alkenyl, optionally substituted alkynyl, acyl, —C(O)-alkyl, —C(O)-alkenyl, or —C(O)-alkynyl; as well as pharmaceutically acceptable esters, amides, salts, solvates, enantiomers, and prodrugs thereof.

In yet another embodiment, the invention comprises compounds that may be prepared according to the methods described herein and have the structure provided in Formula (8)

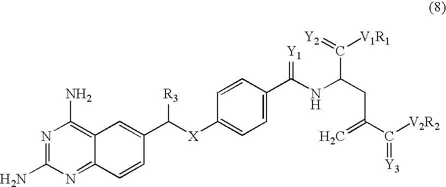

wherein:

X is CHR₈ or NR₈;

Y₁, Y₂, and Y₃ independently are O or S;

V₁ and V₂ independently are O, S, or NZ;

Z is H, optionally substituted alkyl, optionally substituted alkenyl, optionally substituted alkynyl, or alkaryl;

R₁ and R₂ independently are H, optionally substituted alkyl, optionally substituted alkenyl, optionally substituted alkynyl, or alkaryl;

R₃ is H, optionally substituted alkyl, optionally substituted alkenyl, optionally substituted alkynyl, optionally substituted alkoxy, hydroxyl, or halo; and

R₈ is H, optionally substituted alkyl, optionally substituted alkenyl, optionally substituted alkynyl, acyl, —C(O)-alkyl, —C(O)-alkenyl, or —C(O)-alkynyl as well as pharmaceutically acceptable esters, amides, salts, solvates, enantiomers, and prodrugs thereof.

Hereinafter, for ease of understanding by the reader, the present invention is particularly described in reference to a certain, specific embodiments, such as certain, specific crystalline compounds, salt forms, and/or enantiomers of a specific compound, such as the compound of Formula (9), as provided below. Description of the invention in relation to the compound of Formula (9), or specific crystalline forms, salt forms, and/or enantiomers thereof, should not be construed as limiting the scope of the invention. Rather, the invention further encompasses crystalline forms, salt forms, and/or enantiomers of various further specific compounds encompassed by any of the foregoing generic structures (i.e., the compounds encompassed by Formulas (6)-(8)).

In one particular embodiment, the invention comprises compounds that may be prepared according to the methods described herein and have the structure provided in Formula (9).

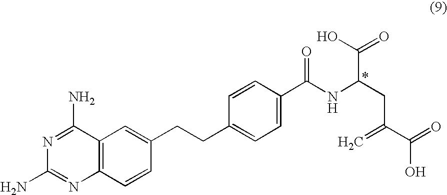

The compound of Formula (9) has been shown to have activity for the treatment of abnormal cellular proliferation, inflammation disorders, and autoimmune diseases. This compound may particularly be known by the name 2-{4-[2-(2,4-diamino-quinazolin-6-yl)-ethyl]-benzoylamino}-4-methylene-pentanedioic acid. The compound may also be known as gamma methylene glutamate 5,8,10-trideaza aminopterin or 5,8-dideaza MDAM. The antifolate compound of Formula (9) is non-polyglutamylatable, non-hydroxylatable, and capable of disrupting folate metabolism. The compound has also shown effectiveness in killing large numbers of human leukemia cells and human solid tumor cells in culture at therapeutically relevant concentrations, and has further shown activity as an anti-inflammatory agent in an animal model of asthma. Unfortunately, the compound suffers from low bioavailability, and the acid form exhibits low solubility, as further described below.

Biologically active variants of the compounds set forth above are particularly also encompassed by the invention. Such variants should retain the general biological activity of the original compounds; however, the presence of additional activities would not necessarily limit the use thereof in the present invention. Such activity may be evaluated using standard testing methods and bioassays recognizable by the skilled artisan in the field as generally being useful for identifying such activity.

According to one embodiment of the invention, suitable biologically active variants comprise one or more analogues or derivatives of the compounds described above. Indeed, a single compound, such as those described above, may give rise to an entire family of analogues or derivatives having similar activity and, therefore, usefulness according to the present invention. Likewise, a single compound, such as those described above, may represent a single family member of a greater class of compounds useful according to the present invention. Accordingly, the present invention fully encompasses not only the compounds described above, but analogues and derivatives of such compounds, particularly those identifiable by methods commonly known in the art and recognizable to the skilled artisan.

The compounds prepared according to the methods of the invention may contain chiral centers, which may be either of the (R) or (S) configuration, or may comprise a mixture thereof. Accordingly, the present invention also includes formation of various stereoisomers, which may include, but are not limited to, enantiomers, diastereomers, racemic mixtures, and combinations thereof. Such stereoisomers can be prepared and separated using conventional techniques, either by reacting enantiomeric starting materials, or by separating isomers of compounds of the present invention. Isomers may include geometric isomers. Examples of geometric isomers include, but are not limited to, cis isomers or trans isomers across a double bond. Other isomers are contemplated among the compounds prepared according to the present invention.

The compound of Formula (9), in particular, is a chiral compound, the chiral center being indicated with an asterisk. Accordingly, the antifolate compound of Formula (9) can exist as two separate enantiomers—either the (R) enantiomer or the (S) enantiomer. Typically, the antifolate compound of Formula (9) exists as a racemic mixture of the two enantiomers.

Various methods are known in the art for preparing optically active forms and determining activity. Such methods include standard tests described herein and other similar tests which are well known in the art. Examples of methods that can be used to obtain optical isomers of the compounds useful according to the present invention include the following:

i) physical separation of crystals whereby macroscopic crystals of the individual enantiomers are manually separated. This technique may particularly be used when crystals of the separate enantiomers exist (i.e., the material is a conglomerate), and the crystals are visually distinct;

ii) simultaneous crystallization whereby the individual enantiomers are separately crystallized from a solution of the racemate, possible only if the latter is a conglomerate in the solid state;

iii) enzymatic resolutions whereby partial or complete separation of a racemate by virtue of differing rates of reaction for the enantiomers with an enzyme;

iv) enzymatic asymmetric synthesis, a synthetic technique whereby at least one step of the synthesis uses an enzymatic reaction to obtain an enantiomerically pure or enriched synthetic precursor of the desired enantiomer;

v) chemical asymmetric synthesis whereby the desired enantiomer is synthesized from an achiral precursor under conditions that produce asymmetry (i.e., chirality) in the product, which may be achieved using chiral catalysts or chiral auxiliaries;

vi) diastereomer separations whereby a racemic compound is reacted with an enantiomerically pure reagent (the chiral auxiliary) that converts the individual enantiomers to diastereomers. The resulting diastereomers are then separated by chromatography or crystallization by virtue of their now more distinct structural differences and the chiral auxiliary later removed to obtain the desired enantiomer;

vii) first- and second-order asymmetric transformations whereby diastereomers from the racemate equilibrate to yield a preponderance in solution of the diastereomer from the desired enantiomer or where preferential crystallization of the diastereomer from the desired enantiomer perturbs the equilibrium such that eventually in principle all the material is converted to the crystalline diastereomer from the desired enantiomer. The desired enantiomer is then released from the diastereomers;

viii) kinetic resolutions comprising partial or complete resolution of a racemate (or of a further resolution of a partially resolved compound) by virtue of unequal reaction rates of the enantiomers with a chiral, non-racemic reagent or catalyst under kinetic conditions;

ix) enantiospecific synthesis from non-racemic precursors whereby the desired enantiomer is obtained from non-chiral starting materials and where the stereochemical integrity is not or is only minimally compromised over the course of the synthesis;

x) chiral liquid chromatography whereby the enantiomers of a racemate are separated in a liquid mobile phase by virtue of their differing interactions with a stationary phase. The stationary phase can be made of chiral material or the mobile phase can contain an additional chiral material to provoke the differing interactions;

xi) chiral gas chromatography whereby the racemate is volatilized and enantiomers are separated by virtue of their differing interactions in the gaseous mobile phase with a column containing a fixed non-racemic chiral adsorbent phase;

xii) extraction with chiral solvents whereby the enantiomers are separated by virtue of preferential dissolution of one enantiomer into a particular chiral solvent; and

xiii) transport across chiral membranes whereby a racemate is placed in contact with a thin membrane barrier. The barrier typically separates two miscible fluids, one containing the racemate, and a driving force such as concentration or pressure differential causes preferential transport across the membrane barrier. Separation occurs as a result of the non-racemic chiral nature of the membrane which allows only one enantiomer of the racemate to pass through.

In particular embodiments, it is preferred for the inventive methods to provide for formation of antifolate compounds in an enantiomerically enriched form, such as a mixture of enantiomers in which one enantiomer is present in excess (given as a mole fraction or a weight fraction). Enantiomeric excess is understood to exist where a chemical substance comprises two enantiomers of the same compound and one enantiomer is present in a greater amount than the other enantiomer. Unlike racemic mixtures, these mixtures will show a net optical rotation. With knowledge of the specific rotation of the mixture and the specific rotation of the pure enantiomer, the enantiomeric excess (abbreviated “ee”) can be determined by known methods. Direct determination of the quantities of each enantiomer present in the mixture (e.g., as a weight %) is possible with NMR spectroscopy and chiral column chromatography.

Chiral compounds are typically prepared as a racemic mixture of the (R) and (S) enantiomers. In one embodiment, the present invention comprises a method for preparing the compound (S)-2-{4-[2-(2,4-diamino-quinazolin-6-yl)-ethyl]-benzoylamino}-4-methylene-pentanedioic acid, which is shown in Formula (10). The compound of Formula (10) is the (S) enantiomer of the compound shown in Formula (9). It is advantageous to have a method for selectively preparing the (S) enantiomer since it can be particularly useful in pharmaceutical compositions in light of its increased activity in comparison to the (R) enantiomer.

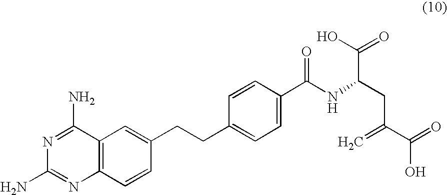

The compounds of the invention may be described in terms of the enantiomeric purity of the compound, which refers to the enantiomeric excess of a specified enantiomer. In one embodiment, a compound may be considered to enantiomerically pure for a specified enantiomer when greater than 50% of the compound present is in the form of the specified enantiomer. In preferred embodiments, the methods of the invention provide antifolate compounds having an enantiomeric purity for the (S) enantiomer of at least about 75%. In other words, at least about 75% of the antifolate compound formed by the method is in the (S) form. In further embodiments, the methods of the invention provide antifolate compounds having an enantiomeric purity for the (S) enantiomer of at least about 80%, at least about 85%, at least about 90%, at least about 95%, at least about 96%, at least about 97%, at least about 98%, at least about 99%, at least about 99.5%, at least about 99.6%, at least about 99.7%, or at least about 99.8%.

The compounds described herein for use in the inventive pharmaceutical compositions can, in certain embodiments, be in the form of an ester, amide, salt, solvate, prodrug, or metabolite provided they maintain pharmacological activity according to the present invention. Esters, amides, salts, solvates, prodrugs, and other derivatives of the compounds of the present invention may be prepared according to methods generally known in the art, such as, for example, those methods described by J. March, Advanced Organic Chemistry Reactions, Mechanisms and Structure, 4^{th} Ed. (New York: Wiley-Interscience, 1992), which is incorporated herein by reference.

Examples of pharmaceutically acceptable salts of the compounds useful according to the invention include acid addition salts. Salts of non-pharmaceutically acceptable acids, however, may be useful, for example, in the preparation and purification of the compounds. Suitable acid addition salts according to the present invention include organic and inorganic acids. Preferred salts include those formed from hydrochloric, hydrobromic, sulfuric, phosphoric, citric, tartaric, lactic, pyruvic, acetic, succinic, fumaric, maleic, oxaloacetic, methanesulfonic, ethanesulfonic, p-toluenesulfonic, benzesulfonic, and isethionic acids. Other useful acid addition salts include propionic acid, glycolic acid, oxalic acid, malic acid, malonic acid, benzoic acid, cinnamic acid, mandelic acid, salicylic acid, and the like. Particular example of pharmaceutically acceptable salts include, but are not limited to, sulfates, pyrosulfates, bisulfates, sulfites, bisulfites, phosphates, monohydrogenphosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxyenzoates, phthalates, sulfonates, xylenesulfonates, phenylacetates, phenylpropionates, phenylbutyrates, citrates, lactates, γ-hydroxybutyrates, glycolates, tartrates, methanesulfonates, propanesulfonates, naphthalene-1-sulfonates, naphthalene-2-sulfonates, and mandelates. An acid addition salt may be reconverted to the free base by treatment with a suitable base.

If a compound of the invention is an acid, the desired salt may be prepared by any suitable method known to the art, including treatment of the free acid with an inorganic or organic base, such as an amine (primary, secondary or tertiary), an alkali metal or alkaline earth metal hydroxide or the like. Illustrative examples of suitable salts include organic salts derived from amino acids such as glycine and arginine, ammonia, primary, secondary and tertiary amines, and cyclic amines such as piperidine, morpholine and piperazine, and inorganic salts derived from sodium, calcium, potassium, magnesium, manganese, iron, copper, zinc, aluminum and lithium.

If a compound useful according to the invention is a base, the desired salt may be prepared by any suitable method known to the art, including treatment of the free base with an inorganic acid, such as hydrochloric acid, hydrobromic acid, sulfuric acid, nitric acid, phosphoric acid and the like, or with an organic acid, such as acetic acid, maleic acid, succinic acid, mandelic acid, fumaric acid, malonic acid, pyruvic acid, oxalic acid, glycolic acid, salicylic acid, pyranosidyl acids such as glucuronic acid and galacturonic acid, alpha-hydroxy acids such as citric acid and tartaric acid, amino acids such as aspartic acid and glutamic acid, aromatic acids such as berizoic acid and cinnamic acid, sulfonic acids such a p-toluenesulfonic acid or ethanesulfonic acid, or the like.

Esters of the compounds according to the present invention may be prepared through functionalization of hydroxyl and/or carboxyl groups that may be present within the molecular structure of the compound. Amides and prodrugs may also be prepared using techniques known to those skilled in the art. For example, amides may be prepared from esters, using suitable amine reactants, or they may be prepared from anhydride or an acid chloride by reaction with ammonia or a lower alkyl amine. Moreover, esters and amides of compounds of the invention can be made by reaction with a carbonylating agent (e.g., ethyl formate, acetic anhydride, methoxyacetyl chloride, benzoyl chloride, methyl isocyanate, ethyl chloroformate, methanesulfonyl chloride) and a suitable base (e.g., 4-dimethylaminopyridine, pyridine, triethylamine, potassium carbonate) in a suitable organic solvent (e.g., tetrahydrofuran, acetone, methanol, pyridine, N,N-dimethylformamide) at a temperature of 0° C. to 60° C. Prodrugs are typically prepared by covalent attachment of a moiety, which results in a compound that is therapeutically inactive until modified by an individual's metabolic system. Examples of pharmaceutically acceptable solvates include, but are not limited to, compounds according to the invention in combination with water, isopropanol, ethanol, methanol, DMSO, ethyl acetate, acetic acid, or ethanolamine.

In one particular embodiment, the invention provides a method for preparing salts of the antifolate compounds described above. In preferred embodiments, the invention provides a method for preparing a salt of the compound according to Formula (9). Accordingly, the salt can be prepared as the racemic form of the compound, as the enantiomerically purified (S) form of the compound, or as the enantiomerically purified (R) form of the compound. Thus, Formula (11) provides compounds that are particularly preferred according to various embodiments of the invention.

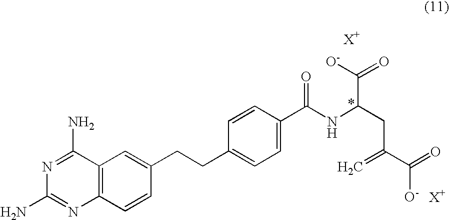

In Formula (11), the asterisk identifies a chiral atom, X⁺ can be any suitable salt-forming counterion, and each X⁺ can be the same or different. In specific embodiments, X⁺ is an alkali metal. In one preferred embodiment, X⁺ is a sodium cation. In another preferred embodiment, X⁺ is a potassium cation. In a specific embodiment, the method of the invention encompasses preparation of a disodium salt according to Formula (11). In still another specific embodiment, the method of the invention encompasses preparation of a dipotassium salt according to Formula (11). Of course, it is understood that other cationic moieties could be used as X⁺ in the compound of Formula (11). Salts of antifolate compounds, such as the disodium salt or dipotassium salt described herein, can be particularly useful in the pharmaceutical compositions in light of their favorable physico-chemical properties.

Antifolate compounds that are in the salt form, in particular a disodium salt or a dipotassium salt, and that are enantiomerically purified for the (S) enantiomer can be particularly useful in pharmaceutical compositions. Accordingly, it is likewise useful to have methods for preparing antifolate compounds that are in an enantiomerically purified salt form. For example, in one embodiment, the invention is directed to a disodium salt or a dipotassium of 2-{4-[2-(2,4-diamino-quinazolin-6-yl)-ethyl]-benzoylamino}-4-methylene-pentanedioic acid that is enantiomerically purified for the (S) enantiomer. The invention also thus encompasses a method of preparing a compound according to Formula (12), wherein X⁺ is sodium or potassium. In one embodiment, the invention encompasses a disodium salt of (S)-2-{4-[2-(2,4-diamino-quinazolin-6-yl)-ethyl]-benzoylamino}-4-methylene-pentanedioic acid. In another embodiment, the invention encompasses a dipotassium salt of (S)-2-{4-[2-(2,4-diamino-quinazolin-6-yl)-ethyl]-benzoylamino}-4-methylene-pentanedioic acid. Preferably, the compound prepared according to the method described herein results in a disodium salt or a dipotassium salt according to Formula (12) that is at least 95% pure for the (S) enantiomer, more preferably at least 97% pure, still more preferably at least 98% pure, even more preferably at least 99% pure, and most preferably at least 99.5% pure for the (S) enantiomer.

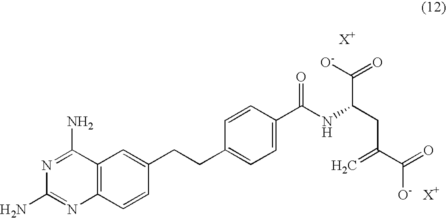

Pharmaceutically active compounds used in pharmaceutical compositions may exist in different forms. For example, the compounds may exist in stable and metastable crystalline forms and isotropic and amorphous forms. The present invention provides methods which encompass the preparation of all such forms.

Crystalline and amorphous forms of the inventive compounds can be characterized by the unique X-ray powder diffraction pattern (i.e., interplanar spacing peaks expressed in Angstroms) of the material. Equipment useful for measuring such data is known in the art, such as a Shimadzu XRD-6000 X-ray diffractometer, and any such equipment can be used to measure the compounds according to the present invention.

In specific embodiments, the invention comprises a method of preparing an antifolate compound in a stable crystalline form, which may be an intermediate compound or the final desired compound. In a specific embodiment, the method comprises the preparation of a compound according to Formula (11) in a stable crystalline form. In a preferred embodiment, the method comprises the preparation of a compound according to Formula (12) in a stable crystalline form, and wherein the compound has an enantiomeric purity for the (S) enantiomer as described herein.

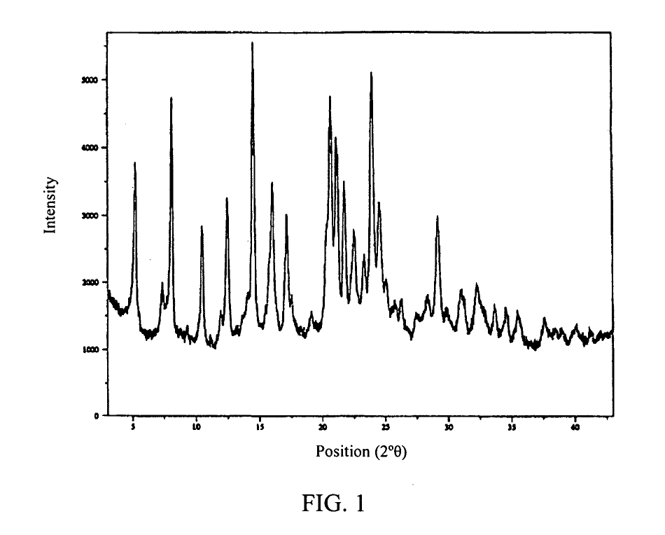

In one embodiment of the invention, an antifolate compound may be a disodium salt characterized by the following approximate X-ray powder diffraction “d-spacing” peaks (i.e., interplanar spacing peaks at 2°θ): 4.8750, 7.3490, 8.1221, 10.5019, 11.8701, 12.4449, 14.5270, 16.0326, 17.1551, 20.6738, 21.1909, 21.7468, 22.5306, 23.2841, 23.9665, 24.4918, 28.3375, 29.1428, 30.8958, 32.2118, 33.5960, 34.5226, and 35.4153. The X-ray powder diffraction pattern for this form of the disodium salt is illustrated in FIG. 1 (which is more fully discussed below in Example 1).

The pharmaceutical compositions of the present invention further include prodrugs and active metabolites of the antifolate compounds of the invention. Any of the compounds described herein can be administered as a prodrug to increase the activity, bioavailability, or stability of the compound or to otherwise alter the properties of the compound. Typical examples of prodrugs include compounds that have biologically labile protecting groups on a functional moiety of the active compound. Prodrugs include compounds that can be oxidized, reduced, animated, deanimated, hydroxylated, dehydroxylated, hydrolyzed, dehydrolyzed, alkylated, dealkylated, acylated, deacylated, phosphorylated, and/or dephosphorylated to produce the active compound. In preferred embodiments, the compounds of this invention possess anti-proliferative activity against abnormally proliferating cells, or are metabolized to a compound that exhibits such activity.

A number of prodrug ligands are known. In general, alkylation, acylation, or other lipophilic modification of one or more heteroatoms of the compound, such as a free amine or carboxylic acid residue, reduces polarity and allows passage into cells. Examples of substituent groups that can replace one or more hydrogen atoms on the free amine and/or carboxylic acid moiety include, but are not limited to, the following: aryl; steroids; carbohydrates (including sugars); 1,2-diacylglycerol; alcohols; acyl (including lower acyl); alkyl (including lower alkyl); sulfonate ester (including alkyl or arylalkyl sulfonyl, such as methanesulfonyl and benzyl, wherein the phenyl group is optionally substituted with one or more substituents as provided in the definition of an aryl given herein); optionally substituted arylsulfonyl; lipids (including phospholipids); phosphotidylcholine; phosphocholine; amino acid residues or derivatives; amino acid acyl residues or derivatives; peptides; cholesterols; or other pharmaceutically acceptable leaving groups which, when administered in vivo, provide the free amine and/or carboxylic acid moiety. Any of these can be used in combination with the disclosed compounds to achieve a desired effect.

### III. Synthesis Methods

Various processes for synthesizing antifolate compounds are disclosed in U.S. Pat. No. 4,996,207, U.S. Pat. No. 5,550,128, Abraham et al. (1991) J. Med. Chem. 34:222-227, and Rosowsky et al. (1991) J. Med. Chem. 34:203-208, all of which are incorporated herein by reference. The present invention provides a synthesis method that is particularly useful for the preparation of antifolates that are in a pharmaceutically useful salt form and/or are in an enantiomerically purified form and/or are in a pharmaceutically stable crystalline form. In certain embodiments, the method of the invention includes the following steps:

1) Providing a starting reagent comprising a benzyl moiety with one or more substituents capable of being cyclized to a 2,4-disubstituted fused aromatic nitrogen-containing heterocycle and an addition site for a coupling reaction;

2) Reacting the starting reagent with a di-substituted benzyl moiety, wherein one substituent is capable of reacting to the free methyl group of the starting reagent and an optionally protected reacting group as a site for a later reaction;

3) Cyclizing the compound of Step 2) to form to a 2,4-disubstituted fused aromatic nitrogen-containing heterocycle coupled to a p-substituted benzyl moiety, the p-substituent being reactive with a group on an optionally substituted glutamate residue;

4) Reacting the compound of Step 3) with a methylene substituted glutamate residue that is further, optionally substituted; and

5) Optionally reacting the compound of Step 4) with reactants suitable to alter the state of the glutamate residue, such as to form an acid or a salt.

The method of synthesis according to the above-described embodiment can be generally described according to Reaction Scheme I provided below.

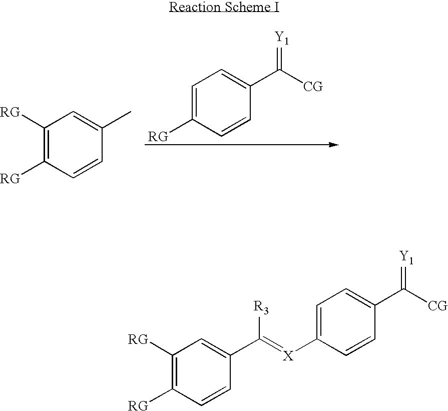

### Starting Reagent

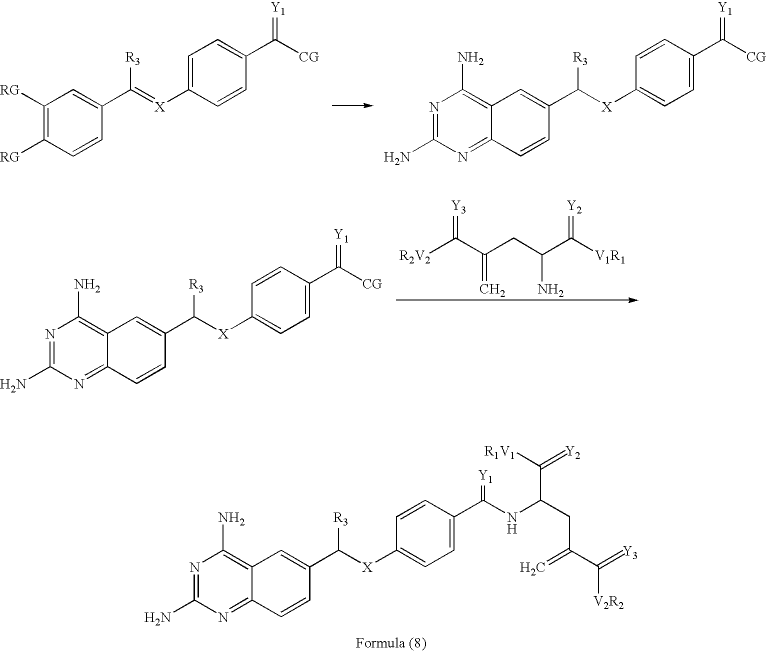

In Reaction Scheme I, RG and CG indicate a reactive group or a temporarily protected reactive group, and X, Y₁, Y₂, Y₃, V₁, V₂, R₁, R₂, and R₃ are as defined above. In a particular embodiment, CG is —O—CH₃, RG is —OH, —CN, —NO₂, or —NH₂, and the cyclizing step is carried out using an amine-containing reactant, such as chloroformamidine hydrochloride.

In specific embodiments, the general nature of Reaction Scheme I can be particularized to arrive at specific antifolate compounds as described above. For example, in Reaction Scheme I, Step 4) can be altered to arrive at an antifolate compound having a desired stereochemistry. In certain embodiments, the methylene substituted glutamate residue can be provided in an enantiomerically purified form. Thus, in Reaction Scheme I, the compound of Formula (8) that is formed by addition of the methylene substituted glutamate residue would incorporate the enantiomerically purified stereochemistry of the methylene substituted glutamate residue. Such reaction, according to one specific embodiment, is provided below.

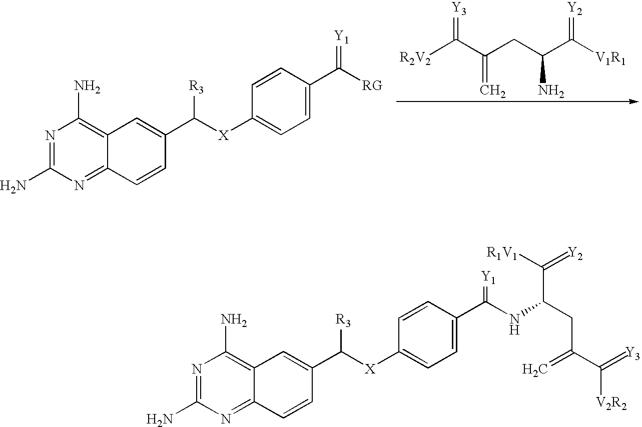

As seen in the above reaction, the chiral carbon of the methylene substituted glutamate moiety is in the optically purified (S) form. Likewise, the formed compound retains this stereochemistry and is also in the enantiomerically purified (S) form. Beneficially, this stereochemistry can be preserved through any further reactions.

The reaction shown above is particularly useful for preparing compounds having a desired stereochemistry. Accordingly, the inventive method can be an independent process for the preparation of enantiomerically purified antifolate compounds. In certain embodiments, the invention can thus comprise providing a 2,4-disubstituted fused aromatic nitrogen-containing heterocycle coupled to a p-substituted benzyl moiety and reacting this compound with an enantiomerically purified methylene substituted glutamate moiety that is optionally further substituted. In one particular embodiment, the reaction step can include the following reaction.

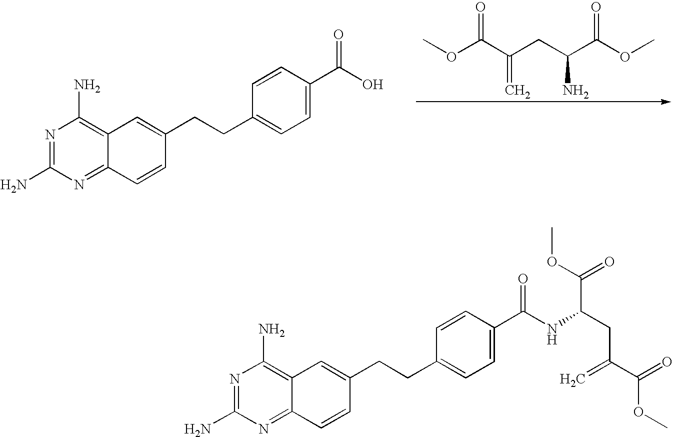

In the above reaction, 4-[2-(2,4-diamino-quinazolin-6-yl)-ethyl]-benzoic acid is reacted with (S)-2-amino-4-methylene-pentanedioic acid dimethyl ester hydrochloride to form (S)-2-{4-[2-(2,4-diamino-quinazolin-6-yl)-ethyl]benzoylamino}-4-methylene-pentanedioic acid dimethyl ester. This compound can undergo further reactions, such as to form the corresponding dioic acid (e.g., through reaction with a base in a suitable solvent, such as acetonitrile). This would particularly result in formation of the compound of Formula (10).

Reaction Scheme I can also be particularized for the formation of various salts of antifolate compounds. For example, Reaction Scheme I could be carried out to result in formation of a compound according to Formula (9), which is 2-{4-[2-(2,4-diamino-quinazolin-6-yl)-ethyl]-benzoylamino}-4-methylene-pentanedioic acid. This diacid structure could then be converted to a corresponding salt, as described above. For example, in specific embodiments, the compound of Formula (9) can be solubilized with an appropriate solvent, such as methanol, and an appropriate base can be added to provide the desired cation. In one embodiment, sodium hydroxide could be added to form the disodium salt. In another embodiment, potassium hydroxide could be added to form the dipotassium salt. The salt compound can then be precipitated by conventional means. Such formation of the di-salt is illustrated below, wherein X⁺ is as defined above.

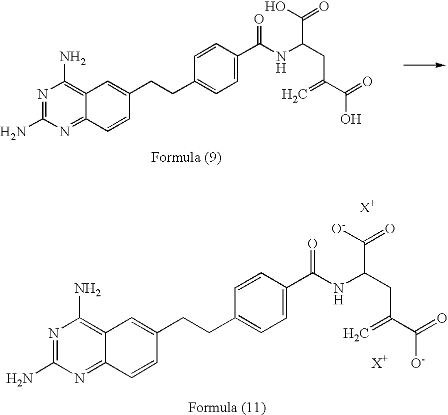

Such a reaction also could be carried out on a compound according to Formula (10) that is the enantiomerically purified form of the dioic acid. Such a reaction would result in the formation of a salt according to Formula (12).

Thus, it is clear that, in further embodiments, the above specific embodiments of Reaction Scheme I can be combined to form salts of the antifolate compounds that are also enantiomerically purified. Thus, in certain embodiments, the invention is directed to a method of preparing an enantiomerically purified antifolate compound in the form of a salt, particularly an alkaline salt. One embodiment of such synthesis is specifically exemplified below in Example 2.

The method of the present invention also provides for the formation of certain intermediate compounds. Such intermediate compounds are stable, crystalline compounds that can be stored for later conversion into an antifolate compound. In particular, formation of the intermediate compounds is useful for increasing product purity by isolating the desired compound apart from any reaction impurities introduced in the upstream process steps.

Accordingly, is certain embodiments, the method of the invention is directed to a method of preparing an intermediate compound useful in the production of antifolate compounds. The method particularly can comprise providing a 2,4-disubstituted fused aromatic nitrogen-containing heterocycle coupled to a benzyl moiety that is p-substituted with a methylene substituted glutamate moiety (that is optionally further substituted) and reacting the compound with a suitable organic counterion. The reaction step in forming the intermediate compound is illustrated below, wherein X, Y₁, Y₂, Y₃, V₁, V₂, R₁, R₂, and R₃ are as defined above.

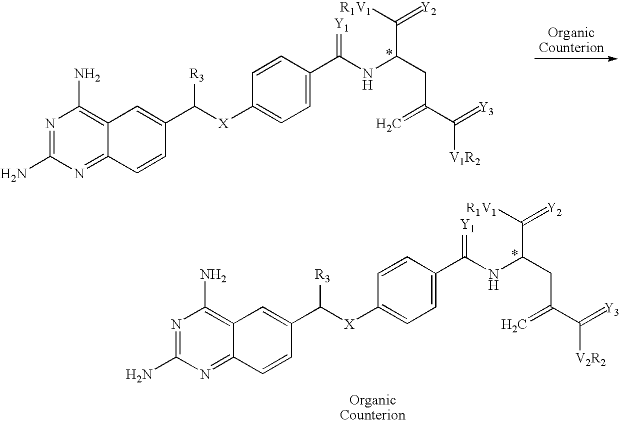

The intermediate compound formed according to the reaction illustrated above can beneficially preserve any specific stereochemistry. For example, the starting compound could be in an enantiomerically purified form, such as the (S) form, and such stereochemistry could be maintained during formation of the intermediate compound, as well as in converting from the intermediate compound to the antifolate compound.

In specific embodiments, the organic counterion used in preparing the intermediate compound can be a linear, branched, or cyclic, optionally substituted, organic moiety having 4 to 20 carbon atoms. In one particular embodiment, the organic counterion is 1-(2-naphthyl)ethylamine. In another embodiment, the organic counterion is a glutamic acid moiety. Moreover, the organic counterion can exhibit specific stereochemistry. For example, in a specific embodiment, the organic counterion can be (R)-(+)-1-(2-naphthyl)ethylamine. For the sake of illustration, a particular reaction using (R)-(+)-1-(2-naphthyl)ethylamine to form the intermediate compound (S)-2-{4-[2-(2,4-diamino-quinazolin-6-yl)-ethyl]-benzoylamino}-4-methylene-pentanedioic acid (R)-(+)-1-(2-naphthyl)ethylamine is shown below. Of course, the reaction shown below could be altered by using a different organic counterion, such as a glutamic acid moiety. Moreover, the starting compound in the reaction below could be variously substituted, as described above.

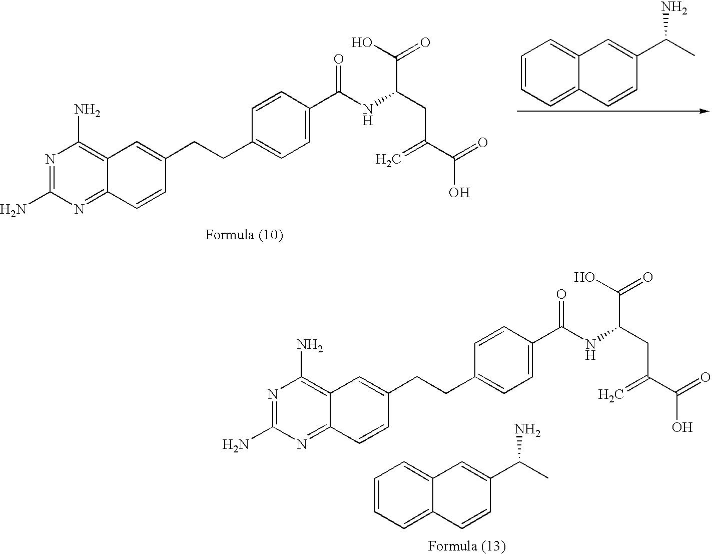

As previously pointed out, the intermediate compound according to Formula (13), or an alternate intermediate compound formed by the reaction generally described above, can be optionally stored for later use and/or subjected to one or more purification steps. Subsequently, the intermediate compound can be converted back into the starting compound. For example, the compound according to Formula (13) can be converted back into the compound of Formula (10), such as by forming a solution of the compound and appropriately adjusting the pH (e.g., through addition of a suitable acid). This is further described below in Example 3.

The synthetic methods of the invention are particularly useful for preparing antifolate compounds for use in pharmaceutical formulations. The compounds prepared according to the inventive methods exhibit increased activity and bioavailability and are thus able to provide therapeutic benefit at reduced total dosages.

The compounds formed according to the inventive methods can be prepared to be in certain pharmaceutically desirable forms, such as esters, amides, salts, solvates, enantiomers, prodrugs, or metabolites. For example, the reaction schemes described particularly illustrate the preparation of salts, but other pharmaceutical forms are also encompassed by the invention. Esters, amides, salts, solvates, prodrugs, and other derivatives can be prepared according to methods generally known in the art, such as, for example, those methods described by J. March, Advanced Organic Chemistry: Reactions, Mechanisms and Structure, 4^{th} Ed. (New York: Wiley-Interscience, 1992), which is incorporated herein by reference.

### IV. Pharmaceutical Compositions

The present invention particularly provides pharmaceutical compositions comprising one or more antifolate compounds as described herein or pharmaceutically acceptable esters, amides, salts, solvates, analogs, derivatives, or prodrugs thereof. Further, the inventive compositions can be prepared and delivered in a variety of combinations. For example, the composition can comprise a single composition containing all of the active ingredients. Alternately, the composition can comprise multiple compositions comprising separate active ingredients but intended to be administered simultaneously, in succession, or in otherwise close proximity of time.

The pharmaceutical compositions can be prepared to deliver one or more antifolate compounds together with one or more pharmaceutically acceptable carriers therefore, and optionally, other therapeutic ingredients. Carriers should be acceptable in that they are compatible with any other ingredients of the composition and not harmful to the recipient thereof. A carrier may also reduce any undesirable side effects of the agent. Non-limiting examples of carriers that could be used according to the invention are described by Wang et al. (1980) J. Parent. Drug Assn. 34(6):452-462, herein incorporated by reference in its entirety.

The pharmaceutical compositions of the invention preferably include an antifolate compound in a therapeutically effective amount, as further described below. In certain embodiments, the amount of antifolate compound in the compositions is based on the overall weight of the composition. For example, in certain embodiments, the pharmaceutical composition comprises an antifolate compound in an amount of about 0.01 mg/g to about 100 mg/g. In further embodiments, the pharmaceutical composition comprises an antifolate compound in an amount of about 0.02 mg/g to about 80 mg/g, about 0.05 mg/g to about 75 mg/g, about 0.08 mg/g to about 50 mg/g, about 0.1 mg/g to about 30 mg/g, about 0.25 mg/g to about 25 mg/g, or about 0.5 mg/g to about 20 mg/g. The amount of drug can also be referenced to a unit dose (e.g., the amount of drug in a single capsule or tablet). The content of the antifolate compound can be referenced to the content of the salt. In other embodiments, even when a salt form is used, the amount of the antifolate compound can be referenced to the content of the free acid present.

Compositions of the present invention may include short-term, rapid-onset, rapid-offset, controlled release, sustained release, delayed release, and pulsatile release compositions, providing the compositions achieve administration of a compound as described herein. See Remington's Pharmaceutical Sciences (18^{th} ed.; Mack Publishing Company, Eaton, Pa., 1990), herein incorporated by reference in its entirety. Pharmaceutical compositions according to the present invention are suitable for various modes of delivery, including oral, parenteral (including intravenous, intramuscular, subcutaneous, intradermal, intra-articular, intra-synovial, intrathecal, intra-arterial, intracardiac, subcutaneous, intraorbital, intracapsular, intraspinal, intrasternal, and transdermal), topical (including dermal, buccal, and sublingual), pulmonary, vaginal, urethral, and rectal administration. Administration can also be via nasal spray, surgical implant, internal surgical paint, infusion pump, or via catheter, stent, balloon or other delivery device. The most useful and/or beneficial mode of administration can vary, especially depending upon the condition of the recipient and the disorder being treated. In preferred embodiments, the compositions of the present invention are provided in an oral dosage form, as more fully described below.

The pharmaceutical compositions may be conveniently made available in a unit dosage form, whereby such compositions may be prepared by any of the methods generally known in the pharmaceutical arts. Generally speaking, such methods of preparation comprise combining (by various methods) the active compounds of the invention with a suitable carrier or other adjuvant, which may consist of one or more ingredients. The combination of the active ingredients with the one or more adjuvants is then physically treated to present the composition in a suitable form for delivery (e.g., shaping into a tablet or forming an aqueous suspension).

Pharmaceutical compositions according to the present invention suitable for oral dosage may take various forms, such as tablets, capsules, caplets, and wafers (including rapidly dissolving or effervescing), each containing a predetermined amount of the active agent. The compositions may also be in the form of a powder or granules, a solution or suspension in an aqueous or non-aqueous liquid, and as a liquid emulsion (oil-in-water and water-in-oil). The active agents may also be delivered as a bolus, electuary, or paste. It is generally understood that methods of preparations of the above dosage forms are generally known in the art, and any such method would be suitable for the preparation of the respective dosage forms for use in delivery of the compositions according to the present invention.

The active compound is included in the pharmaceutical composition in an amount sufficient to deliver to a patient a therapeutic amount of a compound of the invention in vivo in the absence of serious toxic effects. The concentration of active compound in the drug composition will depend on absorption, inactivation, and excretion rates of the drug as well as other factors known to those of skill in the art. It is to be noted that dosage values will also vary with the severity of the condition to be alleviated. It is to be further understood that for any particular subject, specific dosage regimens should be adjusted over time according to the individual need and the professional judgment of the person administering or supervising the administration of the compositions, and that the dosage ranges set forth herein are exemplary only and are not intended to limit the scope or practice of the claimed composition. The active ingredient may be administered at once, or may be divided into a number of smaller doses to be administered at varying intervals of time

A therapeutically effective amount according to the invention can be determined based on the bodyweight of the recipient. For example, in one embodiment, a therapeutically effective amount of one or more compounds of the invention is in the range of about 0.1 μg/kg of body weight to about 5 mg/kg of body weight per day. Alternatively, a therapeutically effective amount can be described in terms of a fixed dose. Therefore, in another embodiment, a therapeutically effective amount of one or more compounds of the invention is in the range of about 0.01 mg to about 500 mg per day. Of course, it is understood that such an amount could be divided into a number of smaller dosages administered throughout the day. The effective dosage range of pharmaceutically acceptable salts and prodrugs can be calculated based on the weight of the parent antifolate to be delivered. If a salt or prodrug exhibits activity in itself, the effective dosage can be estimated as above using the weight of the salt or prodrug, or by other means known to those skilled in the art.

It is contemplated that the compositions of the invention comprising one or more compounds described herein will be administered in therapeutically effective amounts to a mammal, preferably a human. An effective dose of a compound or composition for treatment of any of the conditions or diseases described herein can be readily determined by the use of conventional techniques and by observing results obtained under analogous circumstances. The effective amount of the compositions would be expected to vary according to the weight, sex, age, and medical history of the subject. Of course, other factors could also influence the effective amount of the composition to be delivered, including, but not limited to, the specific disease involved, the degree of involvement or the severity of the disease, the response of the individual patient, the particular compound administered, the mode of administration, the bioavailability characteristics of the preparation administered, the dose regimen selected, and the use of concomitant medication. The compound is preferentially administered for a sufficient time period to alleviate the undesired symptoms and the clinical signs associated with the condition being treated. Methods to determine efficacy and dosage are known to those skilled in the art. See, for example, Isselbacher et al. (1996) Harrison's Principles of Internal Medicine 13 ed., 1814-1882, herein incorporated by reference.

### V. Active Agent Combinations

For use in treating various diseases or conditions, the pharmaceutical compositions of the invention can include the antifolate compounds described above in various combinations. For example, in one embodiment, a pharmaceutical composition according to the invention can comprise a single antifolate compound described herein, such as the compound according to Formula (12). In another embodiment, a pharmaceutical composition according to the invention can comprise two or more antifolate compounds disclosed herein. In still further embodiments, a pharmaceutical composition according to the invention can comprise one or more antifolate compounds described herein with one or more further compounds known to have therapeutic properties. For example, the pharmaceutical compositions described herein can be administered with one or more toxicity-reducing compounds (e.g., folic acid or leucovorin). In further embodiments, the inventive pharmaceutical compositions can be administered with one or more compounds known to be an anti-inflammatory, anti-arthritic, antibiotic, antifungal, or antiviral agent. Such further compounds can be provided as a component of the pharmaceutical composition or can be provided in alternation with the compositions of the invention. In other words, the pharmaceutical compositions of the invention can be administered with the additional active agent(s) in the same composition with the antifolate compounds disclosed herein, or the additional active agent(s) can be administered in a separate delivery form from the pharmaceutical compositions of the invention.

### VI. Articles of Manufacture

The present invention also includes an article of manufacture providing a pharmaceutical compositions comprising one or more antifolate compounds disclosed herein, optionally in combination with one or more further active agents. The article of manufacture can include a vial or other container that contains a composition suitable for use according to the present invention together with any carrier, either dried or in liquid form. In particular, the article of manufacture can comprise a kit including a container with a composition according to the invention. In such a kit, the composition can be delivered in a variety of combinations. For example, the composition can comprise a single dosage comprising all of the active ingredients. Alternately, where more than one active ingredient is provided, the composition can comprise multiple dosages, each comprising one or more active ingredients, the dosages being intended for administration in combination, in succession, or in other close proximity of time. For example, the dosages could be solid forms (e.g., tablets, caplets, capsules, or the like) or liquid forms (e.g., vials), each comprising a single active ingredient, but being provided in blister packs, bags, or the like, for administration in combination.

The article of manufacture further includes instructions in the form of a label on the container and/or in the form of an insert included in a box in which the container is packaged, for the carrying out the method of the invention. The instructions can also be printed on the box in which the vial is packaged. The instructions contain information such as sufficient dosage and administration information so as to allow the subject or a worker in the field to administer the pharmaceutical composition. It is anticipated that a worker in the field encompasses any doctor, nurse, technician, spouse, or other caregiver that might administer the composition. The pharmaceutical composition can also be self-administered by the subject.

### VII. Methods of Treatment

The antifolate compounds of the present invention are particularly useful in the treatment of various conditions wherein disruption of folic acid metabolism is beneficial for treating a symptom of the condition or the condition generally. Accordingly, in further embodiments, the present invention is directed to methods of treating various diseases or conditions. In particular embodiments, the invention provides methods of treating diseases or conditions known or found to be treatable by disruption of folic acid metabolism. In specific embodiments, the invention provides methods of treating conditions, such as abnormal cell proliferation, inflammation (including inflammatory bowel disease), arthritis (particularly rheumatoid arthritis), psoriasis, and asthma.

### EXPERIMENTAL

The present invention will now be described with specific reference to various examples. The following examples are not intended to be limiting of the invention and are rather provided as exemplary embodiments. As used in one or more examples below, “CH-1504” refers to a compound of Formula (9), and such recitation may further define the compound as racemic or “DL” or as a purified enantiomer (i.e., the L-form or D-form). “MTX” refers to methotrexate.

### Example 1

### Salt Screening

The free acid form of the antifolate compound of Formula (9) has a crystalline structure but exhibits poor solubility. A salt screen of this compound was conducted with various pharmaceutically acceptable counterions to analyze aqueous solubility of the formed salts. The counterions used are provided in Table 1. Formed solids suspected of forming salts were analyzed by X-ray powder diffraction (XRPD).

TABLE 1

| Type of | | Type of | |
|---|---|---|---|
| Counterion | Counterion | Counterion | Counterion |
| Mineral acids | Sulfuric | Carboxylic | Benzoic |
| | Hydrochloric | acids | Citric |
| Sulfonic acids | Benzenesulfonic | | Fumaric |
| | 1,2-Ethandisulfonic | | Glycolic |
| | Ethanesulfonic | | Maleic |
| | Isethionic | | DL-malic |
| | Methansulfonic | | Oxalic |
| | 1,5-naphthalenedisulfonic | | Succinic |
| | 2-naphthalenesulfonic | | DL-tartaric |
| | toluenesulfonic | Bases | Ammonium |
| Amino acids | L-arginine | | Calcium |
| | L-lysine | | Potassium |
| | | | Sodium |

Of the various mineral, sulfonic, and carboxylic acids that were tested, crystalline salts were generated using HCl, benzenesulfonic acid, methansulfonic acid, 2-naphalenesulfonic acid, and ethanesulfonic acid. Salt formation was confirmed by ¹H NMR analysis. Solids exhibiting XRPD patterns of mostly amorphous material or with broad, low intensity peaks were obtained using 1,2-ethanedisulfonic acid, 1,5-naphthalenedisulfonic acid, sulfuric acid, and toluenesulfonic acid. No reaction was observed using benzoic acid, citric acid, glycolic acid, maleic acid, DL-malic acid, oxalic acid, fumaric acid, phosphoric acid, succinic acid, or DL-tartaric acid. The XRPD patterns of solids obtained using these acids were similar to the XRPD pattern of the crystalline acid compound of Formula (9).

Of the various bases that were tested, crystalline salts were generated using calcium methoxide. Solids exhibiting XRPD patterns of mostly amorphous material or with broad, low intensity peaks were obtained using ammonium hydroxide and potassium hydroxide. The XRPD pattern of solids obtained from a sodium salt exhibited one peak at about 5.0 2°θ. Salt attempts using L-arginine and L-lysine resulted in solids exhibiting XRPD patterns of mostly amorphous material or with broad peaks.

Hygroscopicity and approximate solubility in aqueous and buffered solutions of ammonium, besylate, calcium, esylate, sulfate, HCl, mesylate, napsylate, potassium, disodium, and tosylate salts were compared. In the hygroscopicity study, the salts were subjected to 75% relative humidity for five days. A new form was obtained from the calcium salt. The ammonium, besylate, esylate, HCl, mesylate, and napsylate salts remained unchanged, but peak shifting was observed with the ammonium and napsylate salts. Tacky or gummy solids or solids not exhibiting birefringence and extinction were obtained from the amorphous sulfate, potassium, disodium, and tosylate salts.

The salts were screened for aqueous solubility as well as solubility in pH 5, 6, and 7 buffer solutions. The solubilities were estimated based on visual observation and do not necessarily reflect the equilibrium solubility. In some samples, when solids remained, the slurry was checked after 1 and 2 days to determine dissolution. The disodium salt exhibited an approximate aqueous solubility of >116 mg/mL, and the potassium salt exhibited an approximate solubility of >98 mg/mL. The remaining salts exhibited an approximate aqueous solubility of 0.4 mg/mL or less.

When tested in a pH 7 (20 mM phosphate) buffer solution, solubility trends were similar to those observed in water. The disodium and dipotassium salts demonstrated the highest solubility (≧32 mg/mL and ≧16 mg/mL, respectively). Solubility of the napsylate salt was ≧1.1 mg/mL, and besylate solubility was ≧2.0 mg/mL. All other salts investigated showed solubilities of <0.2 mg/mL.

Based on the above data, the besylate, napsylate, potassium, and sodium salts were tested in further solubility studies. Approximate solubilities in solutions of pH 5 and 6 were determined. Solubilities were also determined in a pH 7 buffer with increased buffering capacity. Both the besylate and napsylate salts demonstrated a solubility of 0.4 mg/mL at all pH ranges. The disodium salt solubility was ≧37 mg/mL at pH 7 and ≧40 mg/mL at pH 5 and 6. The solubility of the dipotassium salt, measured at pH 7, was ≧16 mg/mL.

The disodium and dipotassium salts were prepared on a larger scale and crystallized in water/IPA and water/acetone. The crystalline disodium salt of the compound of Formula (11), which is designated as Form A (Na), was obtained from both solvent systems. The poorly crystalline dipotassium salt of the compound of Formula (11), which is designated as Form A (K), was obtained from water/IPA. Solids obtained from water/acetone showed slightly improved crystallinity, but the solids still were poorly crystalline.

An abbreviated polymorph screen of the disodium salt of the compound of Formula (9) was conducted, and two crystalline forms were isolated and characterized (designated forms A and B). An amorphous form was also generated. Disodium salt Form A was a crystalline, non-hygroscopic solid containing approximately 4.5 moles of water per mole of the disodium salt of the compound of Formula (11). As described above, disodium salt Form A was a crystalline solid obtained using a water/IPA system or a water/acetone system. Karl Fischer analysis confirmed a water content of 14.8% (equivalent to about 4.75 moles of water per one mole of disodium salt). Hygroscopicity studies showed the material was non-hygroscopic, as determined by visual assessment, when stored at 58% and 75% relative humidity for 14 days, though the XRPD pattern indicated a reduction in crystallinity after storage in 75% RH. VT-XRPD indicated the material lost crystallinity upon heating to 70° C. under a purge of nitrogen. Heating was continued to achieve a temperature of 90° C. Crystallinity was not regained upon cooling to ambient.

Disodium salt Form B was a crystalline hexahydrate obtained from fast evaporation using methanol and trifluoroethanol. Karl Fischer analysis showed 17.5% water (about 6 moles).

The X-ray powder diffraction pattern graph (Cu Kα radiation) of the racemic, disodium salt of the compound of Formula (11)—which is the disodium salt of Form A as described above—is illustrated in FIG. 1, which shows signal intensity at 2°θ. The interplanar spacing peaks of specific 2°θ angles, absolute peak heights, D-spacing, and peak relative intensities of various peaks illustrated in FIG. 1 are particularly provided below in Table 2.

TABLE 2

| Position (2° θ) | Height (Cts) | D-Spacing (A) | Relative Intensity (%) |
|---|---|---|---|
| 4.8750 | 449.49 | 18.10095 | 16.28 |
| 7.3490 | 472.36 | 12.01931 | 17.11 |
| 8.1221 | 2314.59 | 10.87699 | 83.85 |
| 10.5019 | 1101.18 | 8.41690 | 39.89 |
| 11.8701 | 279.44 | 7.44962 | 10.12 |
| 12.4449 | 1386.78 | 7.10681 | 50.24 |
| 14.5270 | 2760.27 | 6.09255 | 100.00 |
| 16.0326 | 1516.46 | 5.52364 | 54.94 |
| 17.1551 | 111.38 | 5.16466 | 40.26 |
| 20.6738 | 2337.29 | 4.29288 | 84.68 |
| 21.1909 | 1587.11 | 4.18930 | 57.50 |
| 21.7468 | 1392.27 | 4.08345 | 50.44 |
| 22.5306 | 777.83 | 3.94315 | 28.18 |
| 23.2841 | 530.22 | 3.81721 | 19.21 |
| 23.9665 | 2401.93 | 3.71003 | 87.02 |
| 24.4918 | 1100.70 | 3.63165 | 39.88 |
| 28.3375 | 349.14 | 3.14692 | 12.65 |
| 29.1428 | 1094.89 | 3.06177 | 39.67 |
| 30.8958 | 359.50 | 2.89192 | 13.02 |
| 32.2118 | 487.65 | 2.77672 | 17.34 |
| 33.5960 | 294.64 | 2.66541 | 10.67 |
| 34.5266 | 355.79 | 2.59567 | 12.89 |
| 35.4153 | 273.34 | 2.53254 | 9.90 |

### Example 2

### Synthesis of (S)-2-{4-[2-(2,4-diamino-quinazolin-6-yl)-ethyl]-benzoylamino}-4-methylene-pentanedioic acid, Disodium Salt

Step 1

6-Nitro-m toluic acid (173 g, 0.96 mol) was dissolved in dichloromethane (2.3 L) and cooled to 5° C. Triethylamine (146 mL, 1.05 mol) was charged over 5 minutes resulting in a clear yellow solution. The internal reaction temperature during the addition was ≦10° C. The solution was cooled to 5° C. and iso-butyl chloroformate (102 mL, 1.05 mol) was charged over 5 minutes with the internal reaction temperature during addition being ≦10° C. The solution was stirred at ambient temperature for 2.5 hours. The solution was cooled to −1° C. and liquid NH₃ (90 g) was added in portions until about pH 11 was achieved, and the mixture was stirred overnight at ambient temperature. The dichloromethane was evaporated. Water (500 mL), aqueous, saturated K₂CO₃ (180 mL), and petroleum ether (boiling pint range 40-60° C., 1.2 L) were added tot eh crude product and stirred for 3 hours at 5° C. The solid was filtered off, washed with petroleum ether (500 mL), and dried to provide 94.3 g product (55% yield) as a beige solid. The overall reaction is shown below.

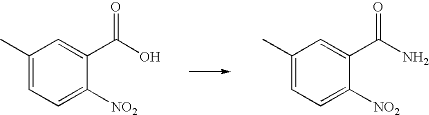

Step 2

A solution of a reaction product as prepared in Step 1 (168 g, 0.93 mol) in dimethylformamide (1 L) was cooled to 0° C. POCl₃ (96 mL, 1.03 mol) was added over 20 minutes with the internal temperature during addition being ≦12° C., and the mixture was stirred for 2 hours at 15° C. until full conversion was achieved. The reaction mixture was cooled to 1° C. and water (1.4 L) was charged over 30 minutes yielding yellow-white slurry, the internal temperature at the end of addition being 18° C. This solution was cooled to 5° C., aqueous NH₃ (1.1 L, 25%) was added until achieving about pH 10, and the mixture was stirred for 1.5 hours at −5° C. The solid was filtered off, washed with water (1 L), and dried furnishing a beige solid product (152 g crude). The solid was suspended in dichloromethane (1 L) and filtered. The filtrate was concentrated yielding pure product as a white solid (114.8 g, 76% yield). The overall reaction is shown below.

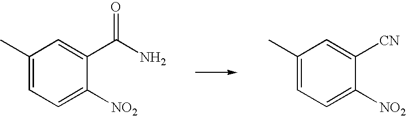

Step 3

A reaction product as prepared in Step 2 (84 g, 0.52 mol) was added to a solution of 4-methoxycarbonylbenzaldehyde (77.4 g, 0.47 mol) in tetrahydrofuran, and the mixture was stirred for 30 minutes at ambient temperature. 1,1,3,3-tetramethylguanidine (60.8 mL, 0.48 mol) was dissolved in THF (600 mL) and charged. The resulting mixture was stirred with heating at reflux for 4.5 days. The reaction was cooled to 25° C., and a mixture of AcOH (84 mL) and water (600 mL) was added drop-wise while stirring. The precipitate was filtered off and mixed with acetone (500 mL). the resulting solid was filtered off, washed with acetone, and dried to yield a yellow solid product (77.1 g, 53% yield). The overall reaction is shown below.

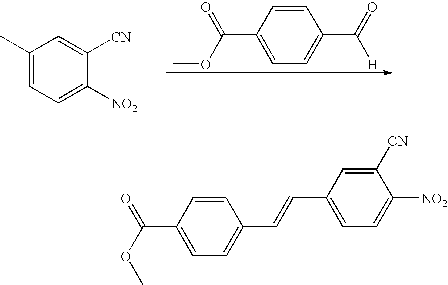

Step 4

A reaction product as prepared in Step 3 (220.8 g, 0.72 mol) was dissolved in tetrahydrofuran (3.7 L) and a catalyst comprising 10% Pd on carbon (2.29 g, 0.02 mol) was added. The atmosphere was exchanged to hydrogen and the reaction mixture was stirred for 40 hours. The mixture was filtered through a CELITE® filter, washed with tetrahydrofuran (1 L) and dichloromethane (1 L), and concentrated to yield a crude product (200 g). The crude product was purified by flash chromatography on silica using dichloromethane and 0.1% triethylamine to yield 83.9 g of purified product. The overall reaction is shown below. As shown, this hydrogenation step results in removal of the central double bond and conversion of the nitro group to an amine.

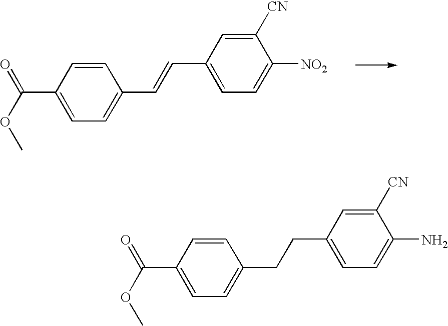

Step 5

A solution of a reaction product as prepared in Step 4 (84 g, 0.4 mol) in sulfolane (635 mL) was heated to 120° C. and chloroformamidine hydrochloride (141 g, 1.23 mol) was added. The reaction mixture was stirred at 140° C. for 1 hour. The mixture was then cooled to 40° C. and water (1.3 L) and 25% aqueous ammonia (280 mL) were added to achieve about pH 9. The formed yellow solid was filtered off and washed with water furnishing crude product (120 g). The overall cyclization reaction is shown below, which results in creation of the amino-substituted quinazoline ring.

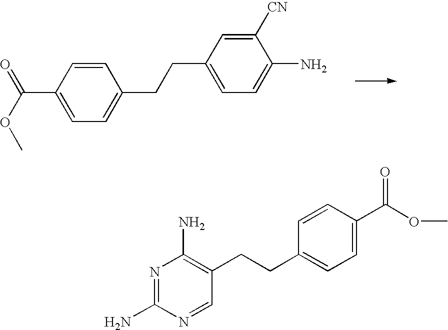

Step 6

Water (250 mL) and NaOH (190 mL, 4M) were added to a solution of tetrahydrofuran (1.3 L) and a reaction product as prepared in Step 5 (120 g). The mixture was stirred at reflux for 4 hours, filtered, and tetrahydrofuran was removed by evaporation. Hydrochloric acid (550 mL, 2M) was added followed by water (500 mL) to achieve about pH 4. The formed yellow solid was filtered off and washed with water (200 mL). The solid was mixed with acetone (500 mL), filtered, and dried under vacuum at 40° C. provide 80 g crude product 4-[2-(2,4-diamino-quinazolin-6-yl)-ethyl]-benzoic acid (86% yield). The overall reaction is shown below. Note that other acids could be used to effect removal of the alkyl protecting group to provide the carboxylic acid.

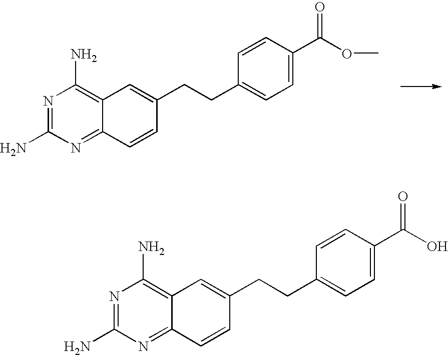

Step 7

4-[2-(2,4-Diamino-quinazolin-6-yl)-ethyl]-benzoic acid from Step 6 (67 g, 0.22 mol), (S)-2-amino-4-methylene-pentanedioic acid dimethyl ester hydrochloride (74 g, 0.33 mol), 1-(3-dimethylaminopropyl)-3-ethyl carbodiimide hydrochloride (140 g, 0.73 mol), 1-hydroxybenzotriazole (3 g, 0.02 mol), 4-dimethylaminopyridine (1 g, 0.01 mol), di-iso-propylethylamine (100 mL), and dimethylformamide (700 mL) were mixed together and heated at 55° C. for 4 hours. The mixture was cooled to room temperature and water (2 L) was added in portions. Precipitation started during the addition of water, and the slurry was equilibrated for 1 hour. The product was filtered and dried on filter paper overnight. The precipitated product of (S)-2-{4-[2-(2,4-diamino-quinazolin-6-yl)-ethyl]-benzoylamino}-4-methylene-pentanediouc acid dimethyl ester (91 g, 86% yield) was inhomogeneous. The overall reaction is shown below.

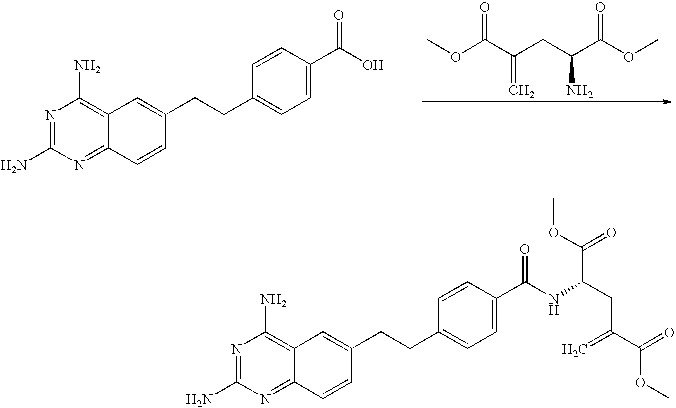

Step 8

(S)-2-{4-[2-(2,4-diamino-quinazolin-6-yl)-ethyl]-benzoylamino}-4-methylene-pentanediouc acid dimethyl ester (90 g, 0.19 mol) was mixed with water (600 mL) and acetonitrile (600 mL). A solution of sodium hydroxide (25 g) in water (200 mL) was added to the mixture at a temperature of 10° C. The reaction mixture was stirred at room temperature for 5 hours. The product was precipitated by addition of hydrochloric acid to pH 5.5. The slurry was equilibrated for 1 hour and filtered. The product was washed with water (200 mL) and dried on the filter to yield 8.25 g (95% yield) of (S)-2-{4-[2-(2,4-diamino-quinazolin-6-yl)-ethyl]-benzoylamino}-4-methylene-pentanedioic acid. The overall reaction is shown below. Other bases could be substituted for sodium hydroxide in the hydrolysis reaction.

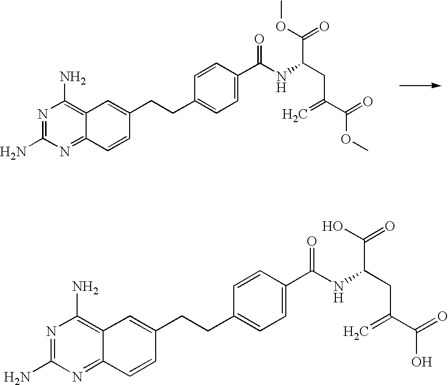

Step 9

(S)-2-{4-[2-(2,4-diamino-quinazolin-6-yl)-ethyl]-benzoylamino}-4-methylene-pentanedioic acid (approximately 33.5 g) was mixed with methanol (300 mL) and the mixture was warmed in a bath (T=50° C.). The pH was adjusted to at least about 10 by addition of 4M aqueous sodium hydroxide (40 mL). Acetone (150 mL) was added to the reaction mixture and a clear solution was obtained. More acetone was added to the reaction mixture in two portions (150 mL and 300 mL) during a period of 1 hour to precipitate the product. The reaction mixture was cooled to room temperature. The product precipitated as an amorphous semi-solid material. The supernatant was decanted from the precipitated product and acetone (200 mL) was added to the residue. The product solidified and was filtered. The obtained product, (S)-2-{4-[2-(3,4-diamino-quinazolin-6-yl)-ethyl]benzolyamino}-4-methylene-pentanedioic acid disodium salt (48 g), was dried and filtered. The overall reaction is shown below. As explained herein, other salt forms could be substituted for the sodium salt shown in this example, such as a dipotassium salt.

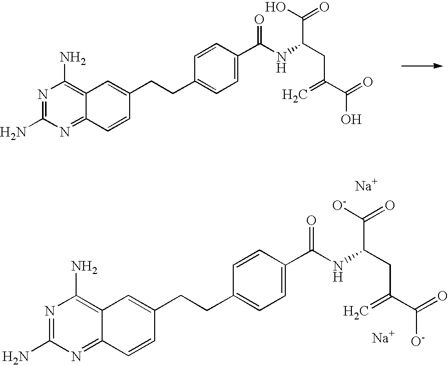

### Example 3

### Synthesis of Stable, Crystalline Intermediate

(S)-2-{4-[2-(2,4-diamino-quinazolin-6-yl)-ethyl]-benzoylamino}-4-methylene-pentanedioic acid (82 g, 0.18 mol) was mixed with water (250 mL). A solution of (R)-(+)-1-(2-naphthyl)ethylamine (37 g, 0.21 mol) in tetrahydrofuran (200 mL) was added to the mixture. The reaction mixture was clear filtered through a CELITE® filter. The product, (S)-2-{4-[2-(2,4-diamino-quinazolin-6-yl)-ethyl]-benzoylamino}-4-methylene-pentanedioic acid (R)-(+)-1-(2-naphthyl)ethylamine (140 g) was precipitated with tetrahydrofuran (3 L), filtered, and dried on the filter for 2 hours. The (R)-(+)-1-(2-naphthyl)ethylamine compound was a stable, crystalline salt. The overall reaction is shown below.

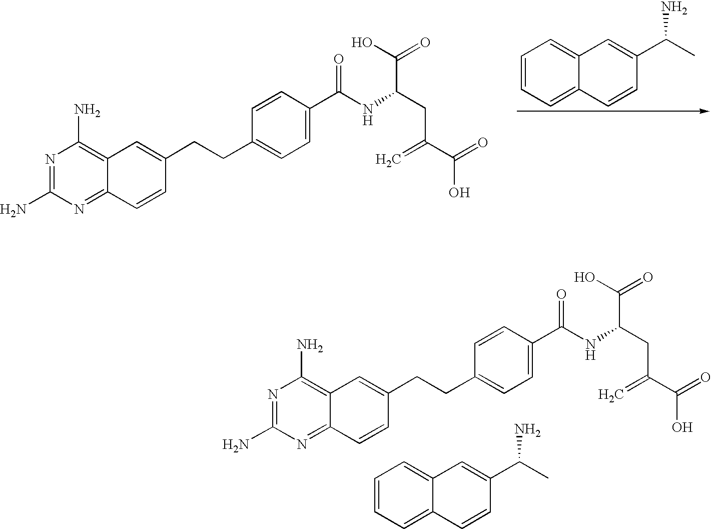

The stable, crystalline (R)-(+)-1-(2-naphthyl)ethylamine compound was converted back to the dioic acid form by mixing with water (300 mL) and adjusting the pH to about 13 by addition of 4 M aqueous sodium hydroxide. The reaction mixture was extracted with dichloromethane (200 mL). The organic phase was discarded and pH of the water phase was lowered to approximately 5 by addition of hydrochloric acid. The precipitated product was filtered, washed with water (200 mL), and dried. The overall reaction is shown below.

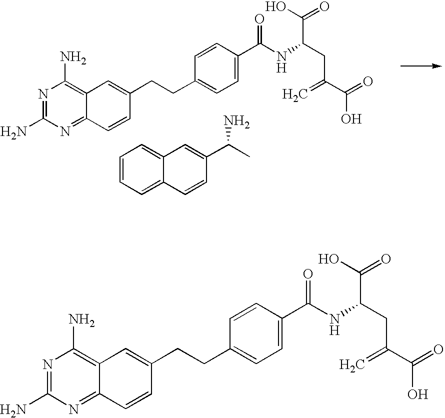

### Example 4

### [³H]MTX Transport Inhibition

Transport of 2 μM [³H]MTX (methotrexate) at 37° by intact CCRF-CEM human T-cell leukemia was assayed by a micro-method utilizing repeated iced saline washes to remove extracellular drug. Such method is disclosed in McGuire J J, et al., Cancer Res 1989; 49:4517-25 and McGuire J J, et al., Cancer Res 2006; 66:3836-44, both of which are incorporated herein by reference in their entirety. The washed cell pellets were solubilized in 1 ml of 0.3% Triton X-100 at 37° C. for 1 hour before transfer to scintillation vials; 10 ml Ecoscint liquid scintillation fluid (National Diagnostics, Atlanta, Ga.) was added and radioactivity was quantitated in a Beckman LS6500 scintillation counter. Intracellular radiolabel was analyzed by HPLC and was shown to be at least 79%, and typically >90%, MTX. Inhibitory potency of analogs was assessed by pre-mixing [³H]MTX with five graded concentrations of analog in 50 μl, such that when diluted to 250 μL with cells the final [³H]MTX concentration was 2 μM (2 μCi/ml) and the compound concentration was as required. Uptake was initiated by addition of 200 μL of cells at ≈2.5×107 cells/ml and 2 aliquots (100 μL) were removed to iced saline and processed at 5 min. Adventitious [³H]MTX binding was determined at 0° C. by adding 200 μl of cells to 25 μl of PBS in a tube and cooling to 0° C. in ice for ≧5 min; following addition of 25 μl of [³H]MTX to achieve a final concentration of 2 μM, 2 aliquots (100 μL) were immediately removed to iced saline and processed. Controls within each experiment showed that [³H]MTX uptake in the absence of analog was linear for 5 min under these conditions; control uptake was typically 12 pmol/107 cells/5 min. IC50 values were determined and are illustrated below in Table 3.

Analytical HPLC was performed on a Rainin Instruments HPLC system using the Dynamax controller and data capture module run on a Macintosh computer, such as described in McGuire J J, et al., J Biol Chem 1990; 265:14073-9, which is incorporated herein by reference in its entirety. C18 reversed-phase (0.4×25 cm; Rainin Microsorb, 5μ) HPLC was performed at 25° C. Detection was by absorbance at 280 and/or 254 nm. For MTX (tr, ≈31.6 min) and 7-OH-MTX (tr, ≈35.2 min) the gradient was from 4-13% ACN in 0.1 M Na-acetate, pH 5.5 over 41 min at 1 ml/min. Compounds did not elute under these conditions; the gradient was adjusted to 4-20% ACN in 0.1 M Na-acetate, pH 5.5 over 41 min.

TABLE 3

| | [³H]MTX transport |
|---|---|
| Compound | inhibition (IC₅₀) (μM) |
| Aminopterin | 1.5 |
| D-MTX | 49 |
| DL-CH-1504 | 1.7 |
| L-CH-1504 | 1.1 |
| D-CH-1504 | 7.6 |

As illustrated in Table 3, the enantiomerically pure form of CH-1504 (L-CH-1504) was shown to be more efficiently transported into cells expressing the reduced folate carrier (RFC) in comparison to the other compounds tested.

### Example 5

### Cell Culture and Growth Inhibition

The human T-lymphoblastic leukemia cell line CCRF-CEM (described in Foley G F, et al., Cancer 1965; 18:522-9) was cultured as described in McCloskey D E, et al., J Biol Chem 1991; 266:6181-7 (both of which are incorporated herein by reference in their entirety) and verified to be negative for Mycoplasma contamination (Mycoplasma Plus PCR primers, Stratagene, La Jolla, Calif.). Growth inhibition of CCRF-CEM cells by continuous (120 hr) drug exposure was assayed as described in Foley and in McGuire J J, et al., Oncology Res 1997; 9:139-47. EC50 values (drug concentration effective at inhibiting cell growth by 50%) were interpolated from plots of percent growth relative to a solvent-treated control culture versus the logarithm of drug concentration by performing a linear regression of the two data points on either side of 50% relative growth and calculating the inhibitor concentration corresponding to 50% relative growth. Results are provided in Table 4.

TABLE 4

| | Growth Inhibition |
|---|---|
| Compounds | (EC₅₀) (nM) |
| MTX | 15 |
| DL-CH-1504 | 8.6 |
| L-CH-1504 | 6.1 |
| D-CH-1504 | 29 |

As illustrated in Table 4, the L-form of CH-1504 exhibits greater growth inhibition as compared to the D-form or the racemic form.

### Example 6

### Plasma Concentration

Racemic CH-1504 was administered once orally to fasted female Lewis rats at a dose of 10 mg/kg (vehicle: 0.11% carboxymethylcellulose/0.45%) TWEEN 80, formulation: suspension). About 750 μL of blood was collected from the jugular vein at 1 and 3 hours after administration. And then, whole of blood was collected from the femoral vein under diethyl ether anesthesia at 6 hours after administration. The collected blood was immediately centrifuged to obtain a plasma sample. L- and D-CH-1504 were extracted from the plasma by solid-phase extraction and were then determined with a LC/MS/MS. Plasma concentrations of L- and D-CH-1504 at each sample are shown in Table 8. Plasma concentrations of L- and D-CH-1504 were not equivalent, showing a difference in pharmacokinetic parameters of each enantiomer. In particular, as illustrated in Table 5, the L-form of CH-1504 exhibited significantly higher plasma concentrations at every collection interval as compared to the D-form, clearly indicating higher bioavailability.

TABLE 5

| | | | Time after | Plasma conc. (ng/mL) | |
|---|---|---|---|---|---|
| | Dose | Animal | Administration | | D-CH- |
| Compound | (mg/kg) | No. | (h) | L-CH-1504 | 1504 |
| Racemic | 10 | YF01 | 1 | 10.6 | 3.12 |
| CH-1504 | | | 3 | 9.82 | 6.79 |
| | | | 6 | 8.53 | 3.91 |
| | | YF02 | 1 | 3.16 | 0.904 |
| | | | 3 | 1.77 | 1.09 |
| | | | 6 | 1.67 | 1.71 |
| | | YF03 | 1 | 3.60 | 1.36 |
| | | | 3 | 5.34 | 3.26 |
| | | | 6 | 10.0 | 5.69 |

### Example 7

### Plasma Concentration

L- or D-CH-1504 was administered once orally to non-fasted female Lewis rats at a dose of 10 mg/kg (vehicle: 0.11% carboxymethylcellulose/0.45% TWEEN 80, formulation: suspension). About 750 μL of blood was collected from the jugular vein at 1 and 3 hours after administration. And then, whole of blood was collected from the femoral vein under diethyl ether anesthesia at 6 hours after administration. The collected blood was immediately centrifuged to obtain a plasma sample. L- and D-CH-1504 were extracted from the plasma by solid-phase extraction and were then determined with a LC/MS/MS. Plasma concentrations of L- and D-CH-1504 at each sample are shown in Table 6. In all samples, isomerization of CH-1504 could not be confirmed by 6 hours after administration of each enantiomer. These results again illustrate significantly higher plasma concentrations for the L-form of the drug.

TABLE 6

| | | | Time after | Plasma conc. (ng/mL) | |
|---|---|---|---|---|---|
| | Dose | Animal | Administration | | D-CH- |
| Compound | (mg/kg) | No. | (h) | L-CH-1504 | 1504 |
| L-CH-1504 | 10 | YF11 | 1 | 118 | BLQ |
| | | | 3 | 59.7 | BLQ |
| | | | 6 | 21.7 | BLQ |
| | | YF12 | 1 | 144 | BLQ |
| | | | 3 | 61.9 | BLQ |
| | | | 6 | 22.7 | BLQ |
| | | YF13 | 1 | 139 | BLQ |
| | | | 3 | 36.8 | BLQ |
| | | | 6 | 22.2 | BLQ |
| D-CH-1504 | 10 | YF21 | 1 | 0.895 | 31.5 |
| | | | 3 | BLQ | 14.3 |
| | | | 6 | BLQ | 8.34 |
| | | YF22 | 1 | BLQ | 20.5 |
| | | | 3 | BLQ | 9.44 |
| | | | 6 | BLQ | 13.6 |
| | | YF23 | 1 | BLQ | 11.0 |
| | | | 3 | BLQ | 8.93 |
| | | | 6 | BLQ | 8.01 |

BLQ: Below limit of quantification (<0.500 ng/mL)

Many modifications and other embodiments of the inventions set forth herein will come to mind to one skilled in the art to which these inventions pertain having the benefit of the teachings presented in the foregoing descriptions. Therefore, it is to be understood that the inventions are not to be limited to the specific embodiments disclosed and that modifications and other embodiments are intended to be included within the scope of the appended claims. Although specific terms are employed herein, they are used in a generic and descriptive sense only and not for purposes of limitation.

That which is claimed:

1. An antifolate compound according to the following formula, 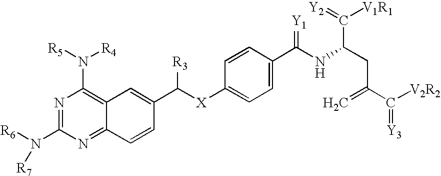
wherein:
X is CHR₈ or NR₈;
Y₁, Y₂, and Y₃ independently are O or S;
V₁ and V₂ independently are O, S, or NZ;
Z is H, optionally substituted alkyl, optionally substituted alkenyl, optionally substituted alkynyl, or alkaryl;
R₁ and R₂ independently are H, optionally substituted alkyl, optionally substituted alkenyl, optionally substituted alkynyl, or alkaryl;
R₃ is H, optionally substituted alkyl, optionally substituted alkenyl, optionally substituted alkynyl, optionally substituted alkoxy, hydroxyl, or halo; and
R₄, R₅, R₆, R₇, and R₈ independently are H, optionally substituted alkyl, optionally substituted alkenyl, optionally substituted alkynyl, acyl, —C(O)-alkyl, —C(O)-alkenyl, or —C(O)-alkynyl; or a pharmaceutically acceptable ester, amide, salt, solvate, enantiomer, or prodrug thereof,
wherein the compound is enantiomerically pure for the (S) enantiomer.

2. The antifolate compound of claim 1, the compound being according to the following formula 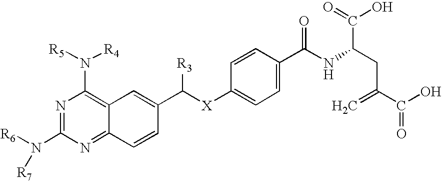
wherein:
X is CHR₈ or NR₈;
R₃ is H, optionally substituted alkyl, optionally substituted alkenyl, optionally substituted alkynyl, optionally substituted alkoxy, hydroxyl, or halo; and
R₄, R₅, R₆, R₇, and R₈ independently are H, optionally substituted alkyl, optionally substituted alkenyl, optionally substituted alkynyl, acyl, —C(O)-alkyl, —C(O)-alkenyl, or —C(O)-alkynyl; or a pharmaceutically acceptable ester, amide, salt, solvate, enantiomer, or prodrug thereof,
wherein the compound is enantiomerically pure for the (S) enantiomer.

3. The antifolate compound of claim 2, the compound being according to the following formula 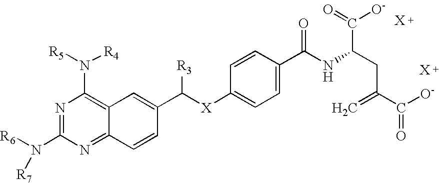
wherein:
X is CHR₈ or NR₈;
R₃ is H, optionally substituted alkyl, optionally substituted alkenyl, optionally substituted alkynyl, optionally substituted alkoxy, hydroxyl, or halo; and
R₄, R₅, R₆, R₇, and R₉ independently are H, optionally substituted alkyl, optionally substituted alkenyl, optionally substituted alkynyl, acyl, —C(O)-alkyl, —C(O)-alkenyl, or —C(O)-alkynyl; or a pharmaceutically acceptable ester, amide, solvate, enantiomer, or prodrug thereof,
wherein the compound is in the form of a crystalline salt that is enantiomerically pure for the (S) enantiomer, and wherein X⁺ is a counterion.

4. The antifolate compound of claim 3, wherein X⁺ is an alkali metal cation.

5. The antifolate compound of claim 3, wherein the antifolate compound is in the form of a disodium salt or a dipotassium salt.

6. The antifolate compound of claim 3, wherein the compound exhibits an enantiomeric purity for the (S) enantiomer of at least about 90%.

7. The antifolate compound of claim 1, the compound being according to the following formula 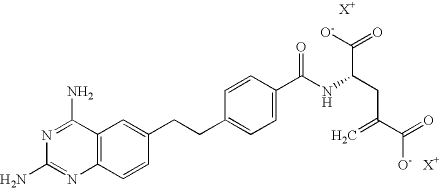
wherein the compound is in the form of a crystalline salt that is enantiomerically pure for the (S) enantiomer, and wherein X⁺ is a counterion.

8. The antifolate compound of claim 3, wherein X⁺ is an alkali metal cation.

9. The antifolate compound of claim 3, wherein the antifolate compound is in the form of a disodium salt.

10. The antifolate compound of claim 3, wherein the antifolate compound is in the form of a dipotassium salt.

11. The antifolate compound of claim 3, wherein the compound exhibits an enantiomeric purity for the (S) enantiomer of at least about 75%.

12. The antifolate compound of claim 11, wherein the compound exhibits an enantiomeric purity for the (S) enantiomer of at least about 90%.

13. The antifolate compound of claim 11, wherein the compound exhibits an enantiomeric purity for the (S) enantiomer of at least about 99%.

14. The antifolate compound of claim 3, wherein the compound is a crystalline, alkali metal salt of (S)-2-{4-[2-(2,4-diamino-quinazolin-6-yl)-ethyl]-benzoylamino}-4-methylene-pentanedioic acid, wherein the compound exhibits an enantiomeric purity for the (S) enantiomer of at least about 95%.

15. The antifolate compound of claim 14, wherein the alkali metal is selected from the group consisting of sodium, potassium, and combinations thereof.

16. The compound (S)-2-{4-[2-(3,4-diamino-quinazolin-6-yl)-ethyl]benzolyamino}-4-methylene-pentanedioic acid disodium salt, wherein the compound is crystalline.

17. The compound of claim 16, wherein the compound exhibits an enantiomeric purity for the (S) enantiomer of at least about 95%.

18. The compound (S)-2-{4-[2-(3,4-diamino-quinazolin-6-yl)-ethyl]benzolyamino}-4-methylene-pentanedioic acid dipotassium salt, wherein the compound is crystalline.

19. The compound of claim 18, wherein the compound exhibits an enantiomeric purity for the (S) enantiomer of at least about 95%.

20. A method of preparing an antifolate compound in the form of a crystalline salt that is enantiomerically pure for the (S) enantiomer, the method comprising:
a) reacting 6-nitro-m toluic acid with triethylamine and iso-butyl chloroformate to form a product according to the following formula; 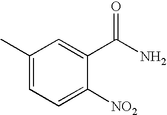
b) reacting the product from step a) with POCl₃ to form a product according to the following formula; 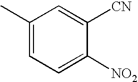
c) reacting the product from step b) with 4-methoxycarbonylbenzaldehyde to form a product according to the following formula; 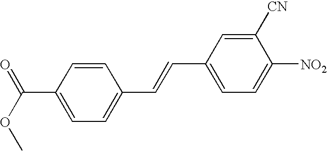
d) reacting the product from step c) with hydrogen in the presence of a catalyst to form a product according to the following formula; 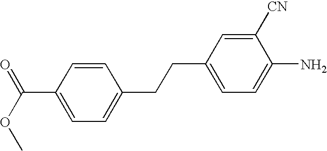
e) cyclizing the product from step d) to form a product according to the following formula: 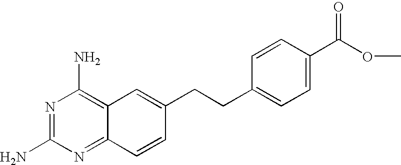
f) reacting the product from step e) with an acid to form a product according to the following formula: 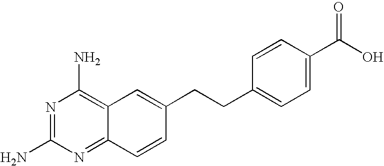
g) reacting the product from step f) with (S)-2-amino-4-methylene-pentanedioic acid dimethyl ester, preferably in the form of a hydrochloride salt, to form a product according to the following formula that is enantiomerically pure for the (S) enantiomer: 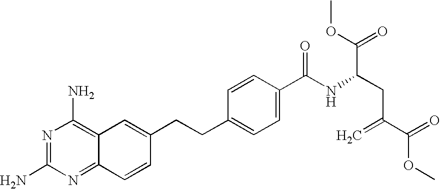
h) reacting the product from step g) with a base to form a product according to the following formula; and 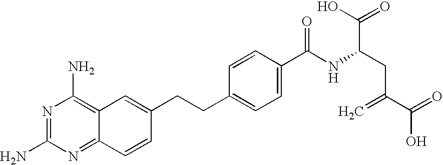
i) reacting the product from step h) with an alkali metal base to form the antifolate compound according to the following formula 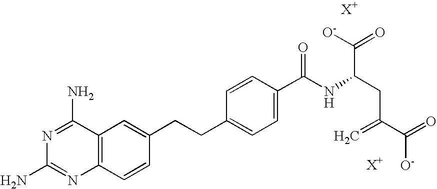
wherein X⁺ is an alkali metal cation, and the compound is in the form of a crystalline salt that is enantiomerically pure for the (S) enantiomer.

21. A method of preparing an antifolate compound according to the following formula 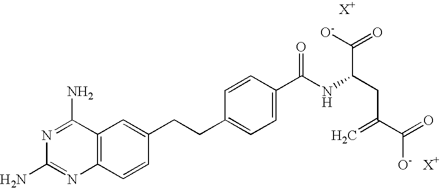
the method comprising the step of reacting 4-[2-(2,4-diamino-quinazolin-6-yl)-ethyl]-benzoic acid with (S)-2-amino-4-methylene-pentanedioic acid dimethyl ester, preferably in the form of a hydrochloride salt, to form an intermediate of (S)-2-{4-[2-(2,4-diamino-quinazolin-6-yl)-ethyl]-benzoylamino}-4-methylene-pentanedioic acid dimethyl ester,
wherein X⁺ is an alkali metal cation and the antifolate compound is in the form of a crystalline salt that is enantiomerically pure for the (S) enantiomer.

22. The method of claim 21, the method further comprising the step of reacting the intermediate (S)-2-{4-[2-(2,4-diamino-quinazolin-6-yl)-ethyl]-benzoylamino}-4-methylene-pentanedioic acid dimethyl ester with a base to form (S)-2-{4-[2-(2,4-diamino-quinazolin-6-yl)-ethyl]-benzoylamino}-4-methylene-pentanedioic acid.

23. The method of claim 22, the method further comprising the step of reacting the (S)-2-{4-[2-(2,4-diamino-quinazolin-6-yl)-ethyl]-benzoylamino}-4-methylene-pentanedioic acid with an alkali metal base to form (S)-2-{4-[2-(3,4-diamino-quinazolin-6-yl)-ethyl]benzolyamino}-4-methylene-pentanedioic acid, alkali metal salt.

24. The method of claim 23, wherein the base is added until achieving a pH of at least about 10.

25. The method of claim 23, the method further comprising precipitating the salt by addition of an organic solvent.

26. The method of claim 22, wherein the prepared antifolate compound exhibits an enantiomeric purity for the (S) enantiomer of at least about 75%.

27. The method of claim 26, wherein the prepared antifolate compound exhibits an enantiomeric purity for the (S) enantiomer of at least about 90%.

28. The method of claim 26, wherein the prepared antifolate compound exhibits an enantiomeric purity for the (S) enantiomer of at least about 99%.

29. A pharmaceutical composition comprising an antifolate compound according to claim 1 and a pharmaceutically acceptable carrier.

30. A method for treating a condition selected from the group consisting of abnormal cell proliferation, inflammation, asthma, and arthritis, said method comprising administering to a subject in need of treatment a compound according to claim 1.
